(19) United States
(12) Patent Application Publication
(10) Pub. No.: US 2025/0345006 A1
(43) Pub. Date: Nov. 13, 2025

(54) HEALTHCARE MANAGEMENT SYSTEM AND HEALTHCARE MANAGEMENT METHOD

(71) Applicants: Murata Manufacturing Co., Ltd., Nagaokakyo-shi (JP); Hyogo Medical University, Nishinomiya-shi (JP)

(72) Inventors: Masahiro SASAKI, Nagaokakyo-shi (JP); Hironori NOTO, Nagaokakyo-shi (JP); Kenta SUZUKI, Nagaokakyo-shi (JP); Ryoya SHIOMOTO, Nagaokakyo-shi (JP); Masanori ASAKURA, Nishinomiya-shi (JP); Masaharu ISHIHARA, Nishinomiya-shi (JP); Kyung-Duk MIN, Nishinomiya-shi (JP); Isamu SUNAYAMA, Nishinomiya-shi (JP); Yoshiyuki ORIHARA, Nishinomiya-shi (JP)

(21) Appl. No.: 19/204,825

(22) Filed: May 12, 2025

(30) Foreign Application Priority Data
May 13, 2024 (JP) 2024-078120
May 13, 2024 (JP) 2024-078121
Mar. 17, 2025 (JP) 2025-042311

(51) Int. Cl. A61B 5/00; A61B 5/024; A61B 5/1455

(52) U.S. Cl. CPC A61B 5/7282; A61B 5/02405; A61B 5/14551; A61B 5/7264

(57) ABSTRACT

A healthcare management system executes a bathing determination process, first and second acquisition processes, a pulse rate data acquisition process, a determination process, and an output process. The bathing determination process determines when a subject starts and finishes bathing. The first acquisition process acquires a first oxygen saturation level that is a blood oxygen saturation level of the subject measured during a first measurement period. The second acquisition process acquires a second oxygen saturation level that is the blood oxygen saturation level of the subject measured during a second measurement period. The pulse rate data acquisition process acquires a pulse rate of the subject. The determination process determines a heart failure-related health condition of the subject based on the first and second oxygen saturation levels and a change in the pulse rate. The output process outputs a determination result of the determination process.

### CROSS-REFERENCE TO RELATED APPLICATIONS

This application is based upon and claims the benefit of priority from Japanese Patent Application No. 2024-078120 filed May 13, 2024, Japanese Patent Application No. 2024-078121 filed May 13, 2024, and Japanese Patent Application No. 2025-042311 filed Mar. 17, 2025, the entire contents of each of which are incorporated herein by reference.

### TECHNICAL FIELD

The present disclosure relates to a healthcare management system and a healthcare management method.

### BACKGROUND

Japanese Laid-Open Patent Publication No. 10-216113 describes a heart failure monitoring device including a blood oxygen saturation level measuring means, an oxygen saturation varying range calculating means, and a heart failure warning means. The blood oxygen saturation level measuring means is configured to measure the blood oxygen saturation level of a subject over time. The oxygen saturation fluctuation range calculating means calculates a change amount of the blood oxygen saturation level measured by the blood oxygen saturation level measuring means. The heart failure warning means outputs a signal indicating a heart failure warning when the change amount exceeds a predetermined reference value.

The blood oxygen saturation level of a subject may change when the subject performs an action. Further, the blood oxygen saturation level of a subject will change differently depending on the type of action performed by the subject. When a heart failure monitoring device such as that described in Japanese Laid-Open Patent Publication No. 10-216113 gives a determination based on the comparison of the change amount of the blood oxygen saturation level with the reference value without taking the above factors into consideration, the occurrence of a change in the health condition related to heart failure may be difficult to detect accurately.

### SUMMARY

In view of the foregoing, according to one exemplary aspect, a healthcare management system is provided that is configured to execute processes including a bathing determination process, a first acquisition process, a second acquisition process, a pulse rate data acquisition process, a determination process, and an output process. The bathing determination process determines when a subject starts bathing and when the subject finishes bathing. The first acquisition process acquires a first oxygen saturation level that is a blood oxygen saturation level of the subject measured during a first measurement period. The first measurement period is at least part of a bathing period from when determined that the subject started bathing to when determined that the subject finished bathing in the bathing determination process. The second acquisition process acquires a second oxygen saturation level that is the blood oxygen saturation level of the subject measured during a second measurement period. The second measurement period is at least part of a non-bathing period from when determined that the subject finished bathing is to when determined that the subject started bathing in the bathing determination process. The pulse rate data acquisition process acquires a pulse rate of the subject. The determination process determines a heart failure-related health condition of the subject based on the first oxygen saturation level, the second oxygen saturation level, and a change in the pulse rate. The output process outputs a determination result of the determination process.

In another exemplary aspect, a healthcare management method is provided that can be implemented by computer executed processes including a bathing determination process, a first acquisition process, a second acquisition process, a pulse rate data acquisition process, a determination process, and an output process. The bathing determination process determines when a subject starts bathing and when the subject finishes bathing. The first acquisition process acquiring a first oxygen saturation level that is a blood oxygen saturation level of the subject measured during a first measurement period. The first measurement period is at least part of a bathing period from when determined that the subject started bathing to when determined that the subject finished bathing in the bathing determination process. The second acquisition process acquires a second oxygen saturation level that is the blood oxygen saturation level of the subject measured during a second measurement period. The second measurement period is at least part of a non-bathing period from when determined that the subject finished bathing to when determined that the subject started bathing in the bathing determination process. The pulse rate data acquisition process acquires a pulse rate of the subject. The determination process determines a heart failure-related health condition of the subject based on the first oxygen saturation level, the second oxygen saturation level, and a change in the pulse rate. The output process outputs a determination result of the determination process.

The above exemplary system and method enable changes in the health condition related to heart failure to be detected accurately.

Other features and aspects will be apparent from the following detailed description and the drawings.

### BRIEF DESCRIPTION OF THE DRAWINGS

FIG. 1 is a diagram illustrating the overall configuration of a healthcare management system in accordance with a first exemplary embodiment.

FIG. 2 is a chart illustrating the average blood oxygen saturation level of a subject having no history of heart failure.

FIG. 3 is a chart illustrating the average blood oxygen saturation level of a subject having a history of heart failure.

FIG. 4 is a sequence diagram illustrating the processing performed by the healthcare management system in accordance with the first exemplary embodiment.

FIG. 5 is a graph illustrating the standard deviation of biological information for a period of 30 minutes after finishing bathing when the subject is a heart failure patient who has no history of rehospitalization.

FIG. 6 is a graph illustrating the standard deviation of biological information for a period of 30 minutes after finishing bathing when the subject is a heart failure patient who has a history of rehospitalization.

FIG. 7 is a sequence diagram illustrating the processing performed by a healthcare management system in accordance with a second exemplary embodiment.

FIG. 8 is a diagram illustrating the overall configuration of a healthcare management system in accordance with a third exemplary embodiment.

FIG. 9 is a sequence diagram illustrating the processing performed by the healthcare management system in accordance with the third exemplary embodiment.

It is noted that throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

This description provides a comprehensive understanding of the methods, apparatuses, and/or systems described. It is noted that modifications and equivalents of the methods, apparatuses, and/or systems described are apparent to one of ordinary skill in the art. Moreover, sequences of operations are exemplary, and may be changed as apparent to one of ordinary skill in the art, with the exception of operations necessarily occurring in a certain order. Descriptions of functions and constructions that are well known to one of ordinary skill in the art may be omitted.

Exemplary embodiments may have different forms and are not limited to the examples described. However, the examples described are thorough and complete, and convey the full scope of the disclosure to one of ordinary skill in the art.

In this specification, “at least one of A and B” refers to each of only A, only B, or both A and B.

A first exemplary embodiment and a second exemplary embodiment of a healthcare management system will now be described with reference to the drawings.

### First Exemplary Embodiment

### Overall Configuration

The healthcare management system 10 is configured to determine a heart failure-related health condition of a subject who is a heart failure patient.

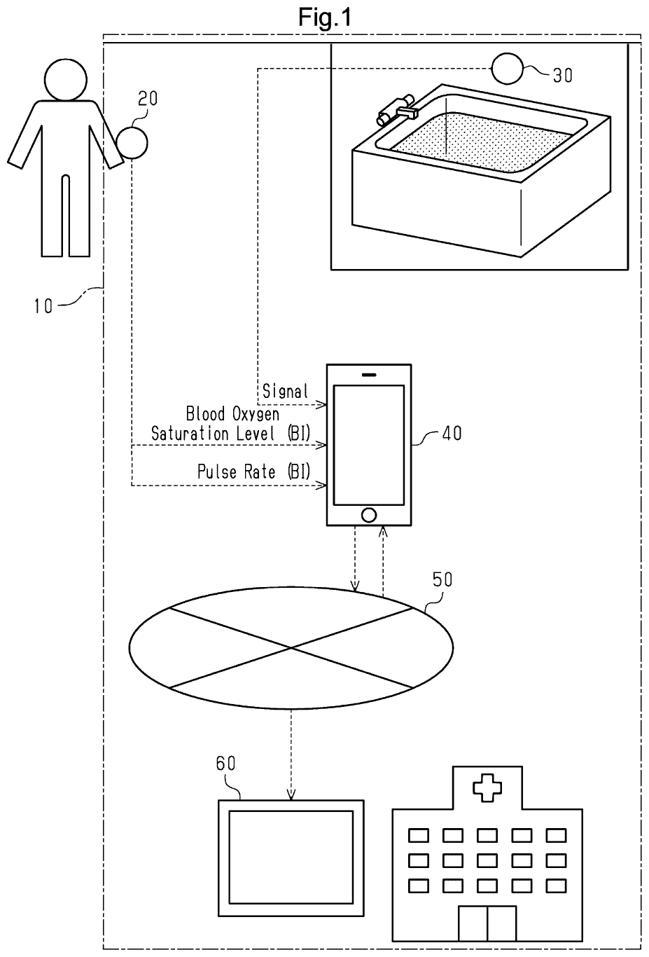

As shown in FIG. 1, the healthcare management system 10 includes a biological sensor 20, a bathing detection sensor 30, a portable terminal 40, a server 50, and an external terminal 60. In an exemplary aspect, each of the portable terminal 40, the server 50, and the external terminal 60 can be computer or similar computing device.

The biological sensor 20 is configured to measure the blood oxygen saturation level and the pulse rate of the subject as biological information BI. In the present embodiment, the biological sensor 20 is a pulse oximeter. Preferably, the pulse oximeter calculates the blood oxygen saturation level and the pulse rate from transmittance of red light and infrared light when irradiating the subject with the red light and the infrared light. Preferably, the biological sensor 20 is of a type attached to a wrist or finger of the subject.

Although not illustrated in the drawings, the biological sensor 20 includes a storage unit and a communication unit. The storage unit and the communication unit may be configured by a single chip or module or be configured by independent chips or modules. The storage unit includes a ROM, which is capable of only reading data, a non-volatile memory, which is capable of reading and writing data, a volatile memory, which is capable of reading and writing data, and the like. The same applies to the following.

The storage unit stores the detected biological information BI. The communication unit is configured to communicate with an external device. The communication unit uses, for example, Bluetooth® for communication in an exemplary aspect. The biological sensor 20 is configured to transmit the biological information BI, which is stored in the storage unit, through the communication unit to the external device.

When the power of the biological sensor 20 is turned on, the biological sensor 20 measures the biological information BI at a predetermined interval of, for example, three minutes. More specifically, the biological sensor 20 can be configured to detect the blood oxygen saturation level and the pulse rate of the patient at the predetermined interval. The biological sensor 20 stores the measured biological information BI in the storage unit. Further, the biological sensor 20 transmits the biological information BI to the portable terminal 40 whenever the biological information BI is measured.

In the present embodiment, the bathing detection sensor 30 is a human presence sensor that detects the presence of a person in a bathroom. The human presence sensor is, for example, a pyroelectric infrared sensor that detects a change in the temperature of a heat source within a detection area. Further, the human presence sensor may be incorporated in a light bulb or the like in the bathroom. Although not shown in the drawings, the bathing detection sensor 30 includes a communication unit. The communication unit is configured to communicate with an external device. The communication unit uses, for example, Bluetooth® for communication in an exemplary aspect.

When detecting a person in the bathroom, the bathing detection sensor 30 transmits a detection signal to the portable terminal 40. When no person is detected in the bathroom, the bathing detection sensor 30 transmits a non-detection signal to the portable terminal 40. The bathing detection sensor 30 is configured to transmit the detection signal and the non-detection signal by communicating with the portable terminal 40 through the communication unit.

In the present embodiment, the portable terminal 40 is a smartphone. The portable terminal 40 is, for example, possessed by the subject. Although not shown in the drawings, the portable terminal 40 includes a storage unit, a first communication unit, and a second communication unit. In the present embodiment, the first communication unit uses Bluetooth® for communication in an exemplary aspect. The second communication unit uses a portable telephone communication network for communication. Accordingly, in the portable terminal 40, the second communication unit has a wider communication range than the first communication range.

The portable terminal 40 is configured to acquire the biological information BI from the biological sensor 20 through the first communication unit. Further, the portable terminal 40 is configured to acquire the detection signal and the non-detection signal from the bathing detection sensor 30 through the first communication unit. The portable terminal 40 is configured to store the biological information BI, the detection signal, and the non-detection signal in the storage unit.

The portable terminal 40 is configured to transmit the biological information BI, the detection signal, and the non-detection signal, which are stored in the storage unit of the portable terminal 40, to the server 50 through the second communication unit.

Although not shown in the drawings, the server 50 includes a control unit, a storage unit (e.g., electronic memory), and a communication unit. More specifically, the control unit is a CPU. The communication unit can be configured to use the portable telephone communication network for communication. The server 50 can be configured to perform various types of processes and algorithms described below by executing programs stored in the storage, for example, the electronic memory. Further, the server 50 is configured to execute a communication process to transmit a signal to the portable terminal 40.

The server 50 is configured to execute a bathing determination process. The bathing determination process determines when a subject started bathing and when the subject finished bathing. More specifically, the server 50 determines that the subject is bathing when the detection signal is acquired. The server 50 determines that the subject finished bathing when acquiring the non-detection signal. As described above, the detection signal is transmitted from the bathing detection sensor 30 when a person is detected in the bathroom. The non-detection signal is transmitted from the bathing detection sensor 30 when no person is detected in the bathroom. Thus, the bathing determination determines that the subject started bathing when the bathing detection sensor 30 detects a person, and after determination that the subject started bathing, determines that the subject finished bathing when the human presence sensor no longer detects the person. The expression of “the server 50 is configured to execute” indicates that the control unit of the server 50 is configured to execute. The same applies to the following description.

The server 50 is configured to execute a first acquisition process. At least part of a bathing period from when the bathing determination process determines that bathing started to when the bathing determination process determines that bathing finished is referred to as the first measurement period according to an exemplary aspect. In the present embodiment, the first measurement period is the entire bathing period. In the first acquisition process, from the segments of biological information BI stored in the storage unit, the server 50 extracts the biological information BI of the subject taken during the first measurement period. Then, the server 50 acquires the average value of the blood oxygen saturation levels included in the biological information BI taken during the first measurement period as a first oxygen saturation level.

Furthermore, the server 50 is configured to execute a second acquisition process. At least part of a non-bathing period from when the bathing determination process determines that bathing finished to when the bathing determination process determines that bathing started is referred to as the second measurement period according to an exemplary aspect. In other words, the second measurement period is at least part of a period before the subject starts bathing and after the subject finishes bathing. In the present embodiment, the second measurement period is a period from when the power of the biological sensor 20 is turned on before bathing to when determined in the bathing determination process that the subject started bathing. In the second acquisition process, from the segments of biological information BI stored in the storage unit, the server 50 extracts the biological information BI of the subject taken during the second measurement period. Then, the server 50 acquires the average value of the blood oxygen saturation levels included in the biological information BI taken during the second measurement period as a second oxygen saturation level.

The server 50 is configured to execute a pulse rate data acquisition process. The pulse rate data acquisition process acquires a pulse rate of the subject. More specifically, from the segments of biological information BI stored in the storage unit, the server 50 extracts the biological information BI of the subject taken during the first measurement period. Then, the server 50 acquires the average value of the pulse rates included in the biological information BI taken during the first measurement period as a first pulse rate. Further, from the segments of biological information BI stored in the storage unit, the server 50 extracts the biological information BI of the subject taken during the second measurement period. Then, the server 50 acquires the average value of the pulse rates included in the biological information BI taken during the second measurement period as a second pule rate.

The server 50 is configured to execute a determination process. The determination process determines a heart failure-related health condition based on the first oxygen saturation level, the second oxygen saturation level, and a change in the pulse rate. For example, in the determination process, the server 50 determines that the heart failure-related health condition of the subject is deteriorating when a first condition and a second condition are both satisfied. Further, in the determination process, the server 50 determines that the heart failure-related health condition of the subject is not deteriorating when one of the first condition and the second condition is not satisfied or when both of the first condition and the second condition are not satisfied.

According to an exemplary aspect, the first condition is that the first oxygen saturation level is less than the second oxygen saturation level and the difference between the first oxygen saturation level and the second oxygen saturation level is greater than or equal to a predetermined first threshold value. Moreover, the second condition is that the first pulse rate is greater than the second pulse rate by a second threshold value or greater. The first threshold value and the second threshold value are set through experiments or the like as values indicating deterioration in the heart failure-related health condition of the subject. The first threshold value is, for example, 4.5%. More specifically, the first threshold value is set within a range from 4% to 8%, inclusive. The second threshold value is, for example, 30/min.

The server 50 is configured to execute an output process. The output process outputs the determination result of the determination process. More specifically, in the output process, the server 50 outputs the determination result of the determination process to the portable terminal 40 and the external terminal 60.

In the present embodiment, the external terminal 60 is a personal computer (PC) set in a hospital. Although not shown in the drawings, the external terminal 60 includes a communication unit. In the present embodiment, the communication unit uses the portable phone communication network for communication. The external terminal 60 is configured to receive a signal from the server 50 through the communication unit.

### First Threshold Value

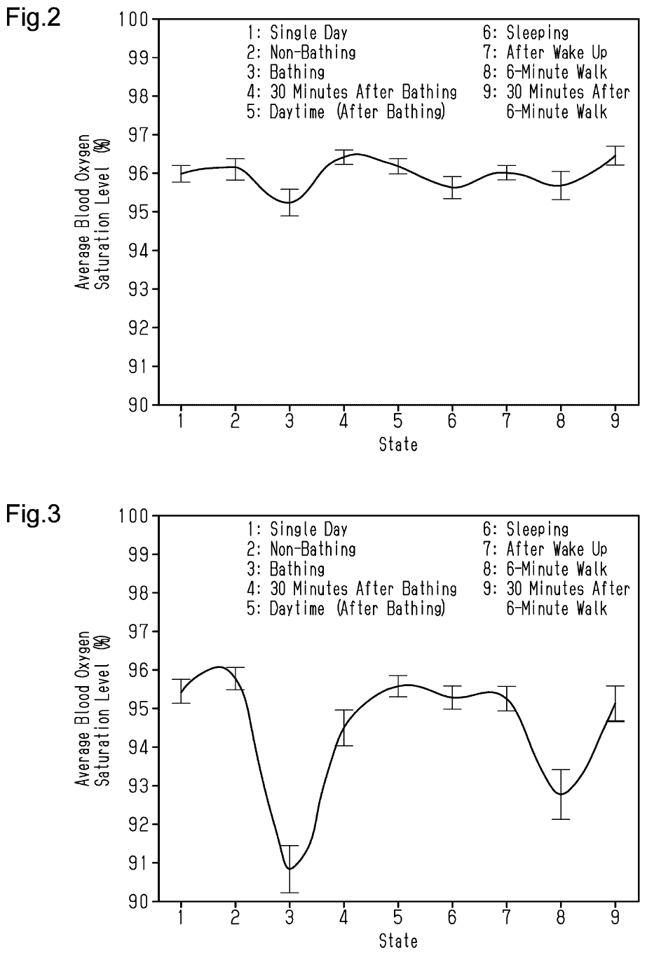

As shown in FIG. 2, the blood oxygen saturation level of a subject who has no history of heart failure was measured in nine states, and the average value for each state was then calculated. The first state is a state averaging a single entire day of the subject. The second state is a non-bathing state. The third state is a bathing state. The fourth state is a state 30 minutes after bathing is finished. The fifth state is a state in daytime after the subject bathes. The sixth state is a state in which the subject is sleeping. The seventh state is a state after the subject wakes up. The eighth state is a state in which the subject is undergoing a six-minute walking test. The ninth state is a state 30 minutes after the six-minute walking test is completed. In the six-minute walking test, the subject walks over level ground for six minutes.

As shown in FIG. 2, the average value of the blood oxygen saturation level in each state was greater than or equal to 95% for the subject who had no history of heart failure. That is, when the subject had no history of heart failure, there was no significant decrease in the blood oxygen saturation level even during bathing.

As shown in FIG. 3, in the nine states, the blood oxygen saturation level of a subject who had a history of heart failure was measured in nine states, and the average value for each state was then calculated. The nine states are the same as the states shown in FIG. 2. As shown in FIG. 3, the average value of the blood oxygen saturation level of the subject was greater than or equal to 95% in the first state, the second state, the fifth state, the sixth state, the seventh state, and the ninth state.

In the eighth state, that is, in a state in which the subject walked for six minutes, the average value of the blood oxygen saturation level of the subject was less than or equal to 93%. In this manner, it became apparent from the experiment that the blood oxygen saturation level decreased when a person having a history of heart failure performed the six-minute walking test.

Further, in the third state, that is, in a state in which the subject was bathing, the average value of the blood oxygen saturation level of the subject was less than or equal to 91%. More specifically, when the subject had a history of heart failure, in addition to when performing the six-minute walking test, the blood oxygen saturation level decreased when the subject was bathing over an extent greater than or equal to the decrease in the six-minute walking test.

Further, in the second state, that is, when the subject was not bathing, the average value of the blood oxygen saturation level was greater than or equal to 95.5%. Thus, the difference between the average blood oxygen saturation level when the subject was bathing and the average blood oxygen saturation level when the subject was not bathing was greater than or equal to 4.5%. Such measurement results indicate that the preferable first threshold value is set within a range from 4% to 8%, inclusive.

### Processing Performed by Healthcare Management System

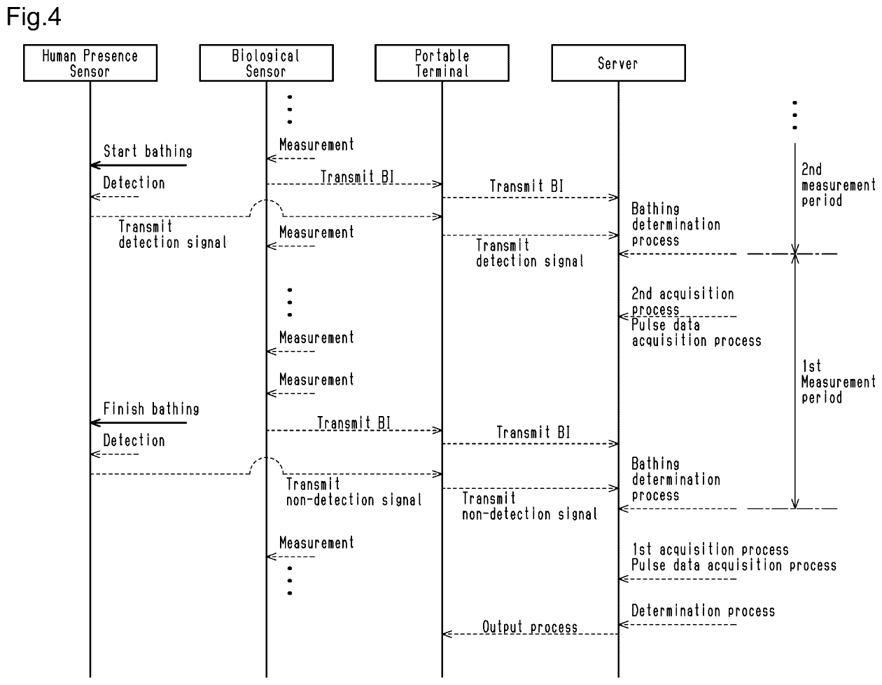

One example of the processing performed by the healthcare management system 10 will now be described with reference to FIG. 4.

The power of the biological sensor 20 attached to the subject is first turned on. The biological sensor 20 can be configured to measure the blood oxygen saturation level and pulse rate of the subject every three minutes as the biological information BI. The biological sensor 20 transmits the biological information BI whenever the biological information BIis measured to the portable terminal 40. FIG. 4 does not show part of the biological information BI transmitted from the biological sensor 20 to the portable terminal 40.

The portable terminal 40 is configured to transmit the biological information BI to the server 50 through the second communication unit whenever the biological information BI is received. The server 50 stores the biological information BI. FIG. 4 does not show part of the biological information BI transmitted from the portable terminal 40 to the server 50.

An example will now be described in which a subject starts bathing at a certain point of time. That is, the subject enters the bathroom. The bathing detection sensor 30 detects that the subject started bathing and transmits a detection signal through the communication unit to the portable terminal 40.

Then, the portable terminal 40 transmits the received detection signal through the second communication unit to the server 50. Upon acquisition of the detection signal from the bathing detection sensor 30, the server 50 executes the bathing determination process. In other words, when the detection signal is acquired, the server 50 determines that the subject started bathing.

When determined that bathing started, the server 50 executes the second acquisition process and the pulse rate data acquisition process. In the second acquisition process, the server 50 acquires the blood oxygen saturation level of the subject measured during the second measurement period as the second oxygen saturation level. The second measurement period is at least part of a non-bathing period from when previously determined that the subject finished bathing to when determined that the subject started bathing. As described above, the second measurement period is a period from when the power of the biological sensor 20 was turned on before bathing to when determined that bathing started in the bathing determination process.

In the second acquisition process, the server 50 first extracts blood oxygen saturation levels from the biological information BI taken during the second measurement period. In the second acquisition process, the server 50 acquires the average value of the extracted blood oxygen saturation levels as the second oxygen saturation level. Further, in the pulse rate data acquisition process of the second acquisition process, the server 50 first extracts pulse rates from the biological information BI taken during the second measurement period. In the pulse rate data acquisition process, the server 50 acquires the average value of the extracted pulse rates as the second pulse rate.

After the server 50 determines that bathing started, the biological sensor 20 measures the blood oxygen saturation level and the pulse rate of the subject every three minutes as the biological information BI in the same manner as before bathing. The biological sensor 20 transmits the biological information BI to the portable terminal 40 whenever the biological information BI is measured. Then, the portable terminal 40 transmits the biological information BI to the server 50 through the second communication unit whenever the biological information BI is received.

An example will now be given in which the subject ends bathing at a certain point of time. That is, the subject leaves the bathroom. The bathing detection sensor 30 detects that the subject finished bathing and transmits a non-detection signal through the communication unit to the portable terminal 40.

Then, the portable terminal 40 transmits the non-detection signal through the second communication unit to the server 50. Upon acquisition of the non-detection signal from the bathing detection sensor 30, the server 50 executes the bathing determination process. In other words, upon acquisition of the non-detection signal, the server 50 determines that the subject finished bathing. The server 50 sets the period from when determined that the subject started bathing to when determined that the subject finished bathing as the first measurement period.

When determined that bathing finished, the server 50 executes the first acquisition process and the pulse rate data acquisition process. More specifically, the server 50 acquires the blood oxygen saturation level and the pulse rate of the subject taken during the first measurement period that is at least part of the bathing period.

In the first acquisition process, the server 50 first extracts the blood oxygen saturation levels from the biological information BI taken during the first measurement period. In the first acquisition process, the server 50 acquires the average value of the extracted blood oxygen saturation levels as the first oxygen saturation level. Further, in the pulse rate data acquisition process, the server 50 extracts the pulse rates from the biological information BI taken during the first measurement period. In the pulse rate data acquisition process, the server 50 acquires the average value of the extracted pulse rates as the first pulse rate.

The server 50 executes the determination process. More specifically, the server 50 determines whether the first oxygen saturation level is less than the second oxygen saturation level by the first threshold value or greater. That is, the server 50 determines whether the first condition, which is described above, is satisfied.

Further, the server 50 determines whether the first pulse rate is greater than the second pulse rate by the second threshold value or greater. That is, the server 50 determines whether the second condition, which is described above, is satisfied.

In the determination process, the server 50 determines that the heart failure-related health condition of the subject is deteriorating when the first condition and the second condition are both satisfied. Further, in the determination process, the server 50 determines that the heart failure-related health condition of the subject is not deteriorating when one of the first condition and the second condition is not satisfied or when both of the first condition and the second condition are not satisfied. In the present embodiment, “deterioration in the heart failure-related health condition of the subject, includes the likelihood of deterioration in addition to actual deterioration.

The server 50 executes the output process. In the output process, the server 50 outputs the determination result of the determination process to the portable terminal 40. For example, when the determination result is “the heart failure-related health condition of the subject is deteriorating,” the server 50 transmits a first signal to the portable terminal 40. Upon receipt of the first signal, the portable terminal 40 can be configured to displayed a text, image, or the like (e.g., on a display device) indicating that the heart failure-related health condition of the subject is deteriorating on the display of the portable terminal 40. Further, upon receipt of the first signal, the portable terminal 40 may show text, image, or the like prompting the subject to visit a hospital in addition to the text, image, or the like indicating that the cardiac arrest-related health condition of the subject is deteriorating on the display of the portable terminal 40. For example, when the determination result is “the heart failure-related health condition of the subject is not deteriorating,” the server 50 transmits a second signal to the portable terminal 40. Upon receipt of the second signal, the portable terminal 40 shows text, image, or the like indicating that the heart failure-related health condition of the subject is not deteriorating on the display of the portable terminal 40. In the output process, the server 50 also outputs the determination result to the external terminal 60. In the same manner as the portable terminal 40, the external terminal 60 outputs the determination result to the display of the external terminal 60 or a display connected to the external terminal 60.

### Technical Advantages of First Exemplary Embodiment

(1-1) In the above embodiment, a heart failure-related health condition of the subject is determined based on the first oxygen saturation level, the second oxygen saturation level, and the pulse rate data. As described above, if the subject has a history of heart failure, the biological information BI changes in a manner differing from a subject having no history of heart failure when bathing and when not bathing. In the above embodiment, in the determination process, the heart failure-related health condition of the subject is determined based on the comparison of the first oxygen saturation level and the second oxygen saturation level and the comparison of the first pulse rate and the second pulse rate. This allows deterioration in a heart failure-related health condition to be detected further accurately.

(1-2) In the above embodiment, in the determination process, when the first oxygen saturation level is less than the second oxygen saturation level and the difference of the first oxygen saturation level and the second oxygen saturation level is greater than or equal to the predetermined first threshold value, the server 50 determines that the heart failure-related health condition of the subject is deteriorating. That is, the server 50 determines further accurately whether a decrease in the blood oxygen saturation level during bathing is caused by deterioration in the heart failure-related health condition.

(1-3) In the above embodiment, the first threshold value is set within a range of 4% to 8%, inclusive. The range of the first threshold value allows deterioration of the heart failure-related health condition caused by bathing to be evaluated further accurately.

(1-4) In the above embodiment, in the determination process, when the first pulse rate is greater than the second pulse rate by the second threshold value or greater, the server 50 determines that the heart failure-related health condition of the subject is deteriorating. That is, the server 50 determines further accurately whether a change in the pulse rate before and after bathing is caused by deterioration in the heart failure-related condition.

(1-5) In the above embodiment, in the output process, the server 50 outputs the determination result of the determination process to the portable terminal 40. This allows the subject to check the heart failure-related health condition.

(1-6) In the above embodiment, in the bathing determination process, the server 50 determines that the subject started bathing when the bathing detection sensor 30 shifts from a state in which no person is detected to a state in which a person is detected. Further, after determining that the subject started bathing, the server 50 determines that the subject finished bathing when the bathing detection sensor 30 shifts from a state in which the person is detected to a state in which the person is no longer detected. In this manner, the bathing determination process is automatically performed. Since there is no need to remember when to input data, determinations in the determination process may be given with further accuracy.

### Second Exemplary Embodiment

The second exemplary embodiment of the healthcare management system will now be described. Elements that are the same as the corresponding elements in the first embodiment will not be described or be described briefly.

When the power of the biological sensor 20 is turned on, the biological sensor 20 measures the biological information BI at a predetermined interval of, for example, every tens of seconds. More specifically, the biological sensor 20 detects the blood oxygen saturation level and the pulse rate of the subject at a predetermined interval. The biological sensor 20 stores the measured biological information BI in the storage unit. The biological sensor 20 transmits the biological information BI to the portable terminal 40 whenever the biological information BI is measured.

The server 50 is configured to execute the first acquisition process. At least part of a bathing period from when the bathing determination process determines that bathing started to when the bathing determination process determines that bathing finished is referred to as the first measurement period according to an exemplary aspect. In the present embodiment, the first measurement period is the entire bathing period. In the first acquisition process, from the segments of biological information BI stored in the storage unit, the server 50 extracts the biological information BI of the subject taken during the first measurement period. Then, in the first acquisition process, the server 50 acquires the average value of the blood oxygen saturation levels included in the biological information BI taken during the first measurement period as a first oxygen saturation level.

The server 50 is configured to execute the second acquisition process. At least part of a non-bathing period from when the bathing determination process determines that bathing finished to when the bathing determination process determines that bathing started is referred to as the second measurement period according to an exemplary aspect. In other words, the second measurement period is at least part of a period before the subject starts bathing and after the subject finishes bathing. In the present embodiment, the second measurement period is a period from when determined that the subject finished bathing to when the power of the biological sensor 20 is turned off. In the second measurement period includes a time point after a predetermined specific time elapses from when determined that the subject finished bathing in the bathing determination process. The predetermined specific time is, for example, 30 minutes. In the second acquisition process, from the biological information BI of the subject taken during the second measurement period, the server 50 extracts the biological information BI taken at the time point closest to 30 minutes after determined that the subject finished bathing. Then, in the second acquisition process, the server 50 acquires the blood oxygen saturation level included in the extracted biological information BI as the second oxygen saturation level.

The server 50 is configured to execute the pulse rate data acquisition process. The pulse rate data acquisition process acquires a pulse rate of the subject. More specifically, in the pulse rate data acquisition process, the server 50 extracts the biological information BI taken during a pre-bathing period before determination that the subject started bathing in the bathing determination process. Then, in the pulse rate data acquisition process, the server 50 acquires the average value of the pulse rates included in the biological information BI taken during the pre-bathing period. When a predetermined period elapses after detecting that the subject finished bathing in the bathing determination process, the server 50 resets the determination. The server 50 sets the period from the resetting to when next determined that the subject started bathing as the pre-bathing period. Further, in the pulse rate data acquisition process, the server 50 extracts the biological information BI taken at the time point closest to after a predetermined specified time elapses from when determined that the subject finished bathing in the bathing determination process. Then, the server 50 acquires the pulse rate included in the extracted biological information BI as a post-bathing pulse rate. In the present embodiment, the specified time is 10 minutes. The expression after the specified time elapses includes the time point at which the specified time elapses and any time point after the specified time elapses.

The server 50 is configured to execute a determination process. The determination process determines a heart failure-related health condition of the subject based on the first oxygen saturation level, the second oxygen saturation level, and a change in the pulse rate. For example, in the determination process, the server 50 determines that the heart failure-related health condition of the subject is deteriorating when a first condition and a second condition are both satisfied. Further, in the determination process, the server 50 determines that the heart failure-related health condition of the subject is satisfactory when one of the first condition and the second condition is not satisfied or when both of the first condition and the second condition are not satisfied.

The first condition is that a post-bathing pulse rate is greater than a pre-bathing pulse rate by a predetermined reference value or greater. The second condition is that a value obtained by subtracting the first oxygen saturation level from the second oxygen saturation level is less than or equal to a predetermined value set within a range of 3% to 4%, inclusive. The reference value and the predetermined value are set through experiments or the like as values indicating deterioration in the heart failure-related health condition of the subject.

### Reference Value

As described above and shown in FIG. 2, the average value of the blood oxygen saturation level in each state was 95% or greater for the subject who had no history of heart failure.

As shown in FIG. 3, the average value of the blood oxygen saturation level of the subject was greater than or equal to 95% in the first state, the second state, the fifth state, the sixth state, the seventh state, and the ninth state for the subject who had a history of heart failure.

As described above, in the third state, that is, in a state in which the subject was bathing, the average value of the blood oxygen saturation level of the subject was less than or equal to 91%. Further, in the fourth state, that is, in a state 30 minutes after the subject finished bathing, the blood oxygen saturation level of the subject was in a range from 94% to 95%, inclusive. Thus, the difference between the average value of the blood oxygen saturation level when the subject was bathing and the average value of the blood oxygen saturation level 30 minutes after the subject finished bathing was within a range from 3% to 4%, inclusive. Such measurements indicate that the preferable reference value is within a range of 4% to 8%, exclusive.

### Specified Time Point

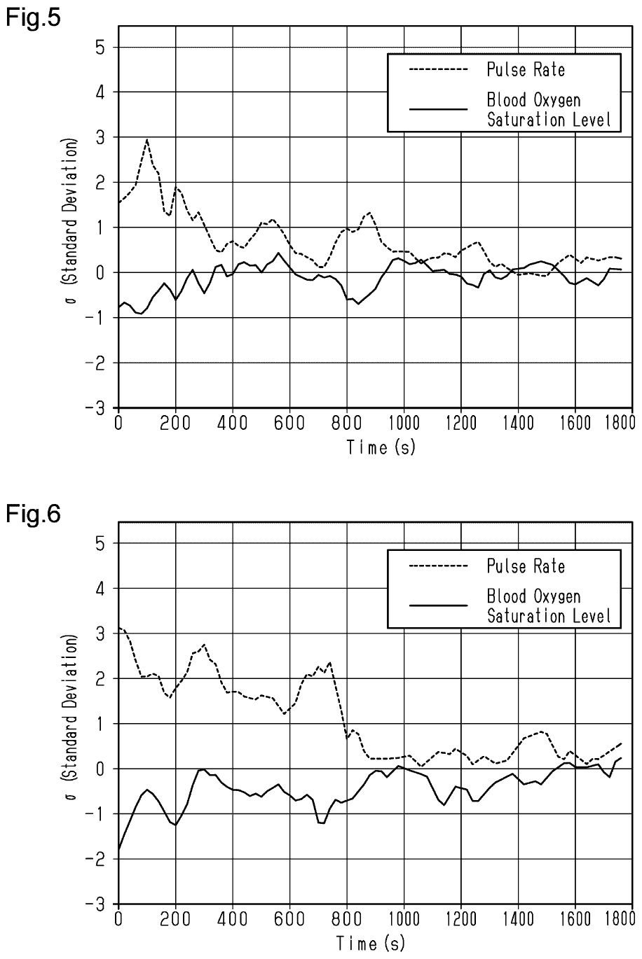

The distribution of the pulse rate and the distribution of the blood oxygen saturation level for several tens of minutes before bathing was obtained from a patient, or subject, having no history of heart failure-related rehospitalization. Further, as shown in FIG. 5, for the same patient, or subject, the deviation of the pulse rate and the blood oxygen saturation level taken during a period of 30 minutes after bathing from the average value of the distribution of the pulse rate and the average value of the distribution of the blood oxygen saturation level taken before bathing was calculated as the standard deviation. In FIG. 5, the broken lines show the transition of the pulse rate, and the solid lines show the transition of the blood oxygen saturation level. The reference value was set as the average value of the measurements taken from the subject over several tens of minutes before bathing. In the graph of FIG. 5, the standard deviation of the pulse rate that is less than 0 indicates that the pulse rate is less than the average value taken before bathing. The standard deviation of the pulse rate that is greater than 0 indicates that the pulse rate was greater than the average value taken before bathing. Further, the standard deviation of the blood oxygen saturation level that is less than 0 indicates that the blood oxygen saturation level was less than the average value taken before bathing. The standard deviation of the blood oxygen saturation level that is greater than 0 indicates that the blood oxygen saturation level was greater than the average value taken before bathing.

As shown in FIG. 5, when the subject is a patient having no history of heart failure-related rehospitalization, the standard deviation of the pulse rate fluctuated and approached 0 during a period from 0 to 600 seconds. The standard deviation of the pulse rate became less than 1 at approximately 600 seconds, that is, approximately, 10 minutes. More specifically, when the subject is a heart failure patient having no history of rehospitalization, the pulse rate taken 10 minutes after the subject finishes bathing was not greatly deviated from the pulse rate taken before bathing.

When the subject is a patient having no history of heart failure-related rehospitalization, the standard deviation of the blood oxygen saturation level fluctuated and approached 0 during a period from 0 to 600 seconds. The standard deviation of the blood oxygen saturation level became close to 0 at approximately 600 seconds, that is, approximately, 10 minutes. In the case of this subject, the standard deviation of the blood oxygen saturation level also became close to 0 at approximately 1200 seconds, that is, approximately, 20 minutes. More specifically, when the subject is a heart failure-related patient having no history of rehospitalization, the blood oxygen saturation levels taken 10 minutes and 20 minutes after bathing was finished were not greatly deviated from the blood oxygen saturation level taken before bathing. In the case of this subject, the standard deviation of the blood oxygen saturation level started from a negative value and became a positive value within a period of 0 to 600 seconds.

As shown in FIG. 6, for a subject who is a patient having a history of heart-failure related hospitalization, the standard deviations of the pulse rate and the blood oxygen saturation level taken during a period of 30 minutes after bathing were calculated in the same manner as the example shown in FIG. 5. In FIG. 6, the broken lines show the transition of the pulse rate, and the solid lines show the transition of the blood oxygen saturation level. The reference value was set in the same manner as the example shown in FIG. 5. When the subject is a heart failure-related patient having a history of rehospitalization, the standard deviation of the pulse rate was greater than or equal to 1 for approximately 600 seconds, that is, for approximately 10 minutes. In other words, when the subject is a heart failure-related patient having a history of rehospitalization and the average value of the pulse rate before bathing is set as the reference, the pulse rate taken 10 minutes after the subject finished bathing was relatively greater than the reference. In this manner, when comparing the pulse rate taken 10 minutes after bathing was finished with the pulse rate before bathing and a heart failure-related patient has a history of rehospitalization thus tending to show heart-failure symptoms, the pulse rate taken 10 minutes after bathing finished was greater than the pulse rate before bathing.

When the subject is a patient having a history of heart failure-related rehospitalization, the standard deviation of the blood oxygen saturation level became about minus 0.5 at approximately 600 seconds, that is, approximately. 10 minutes. In the case of this subject, the standard deviation of the blood oxygen saturation level was also a negative value at approximately 1200 seconds, that is, approximately 20 minutes. In this manner, when a heart failure-related patient has a history of rehospitalization and thus tending to show heart failure symptoms, the blood oxygen saturation levels taken 10 minutes after and 20 minutes after bathing was finished are less than the blood oxygen saturation level taken before bathing.

In the case of this subject, the standard deviation of the blood oxygen saturation level was negative throughout the period of 0 to approximately 600 seconds. In other words, when the subject is a heart failure-related patient having a history of rehospitalization and the average value of the blood oxygen saturation level before bathing is set as a reference, the blood oxygen saturation level was less than the reference throughout the period of 10 minutes after bathing was finished.

### Processing Executed by Healthcare Management System

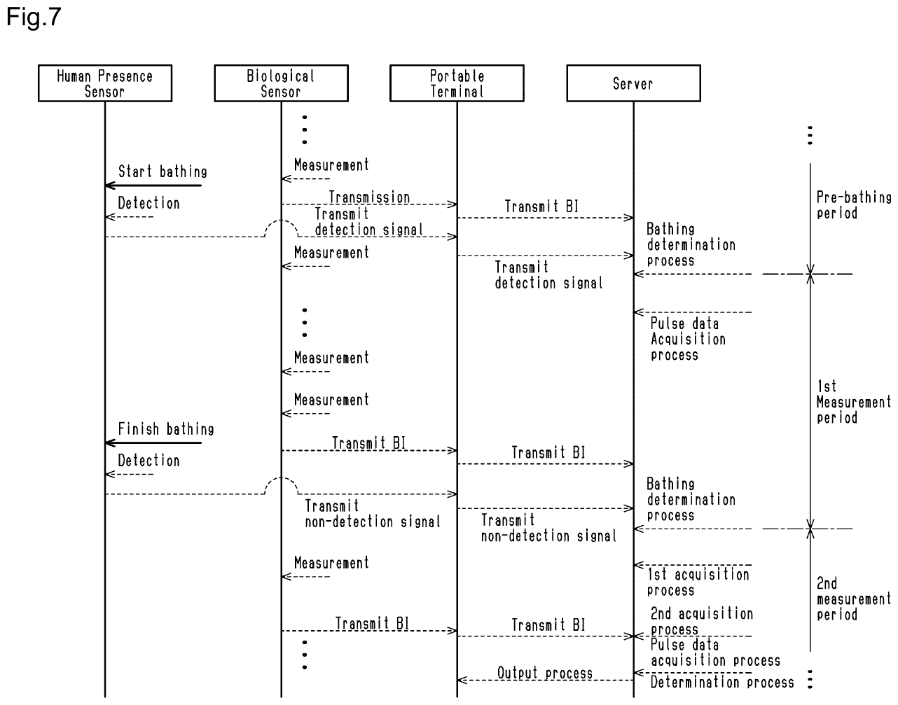

With reference to FIG. 7, one example of the processing performed by the healthcare management system 10 will now be described. The portable terminal 40, the server 50, and the external terminal 60 cooperate with one another as computers to execute processes and implement a healthcare management method including the processes described above.

The power of the biological sensor 20 attached to the subject, who is a heart failure-related patient, is first turned on. The biological sensor 20 measures the blood oxygen saturation level and the pulse rate of the subject every several tens of seconds as the biological information BI. The biological sensor 20 transmits the biological information BI whenever the biological information BI is measured to the portable terminal 40. FIG. 7 does not show part of the biological information BI transmitted from the biological sensor 20 to the portable terminal 40.

Then, the portable terminal 40 transmits the biological information BI to the server 50 through the second communication unit whenever the biological information BI is received. The server 50 stores the biological information BI. FIG. 7 does not show part of the biological information BI transmitted from the portable terminal 40 to the server 50.

An example will now be described in which a subject starts bathing at a certain point of time. That is, the subject enters the bathroom. The bathing detection sensor 30 detects that the subject started bathing and transmits a detection signal through the communication unit to the portable terminal 40.

Then, the portable terminal 40 transmits the received detection signal through the second communication unit to the server 50. Upon acquisition of the detection signal from the biological sensor 20, the server 50 executes the bathing determination process. In other words, when the detection signal is acquired, the server 50 determines that the subject started bathing.

When determined that bathing started, the server 50 executes the pulse rate data acquisition process. In other words, the server 50 acquires the pulse rates taken during a pre-bathing period, which is before determined that the subject started bathing, as a pre-bathing pulse rate. More specifically, the server 50 extracts the biological information BI of the subject that is taken during the pre-bathing period and stored in the storage unit. Then, the server 50 acquires the average value of the pulse rates included in the extracted biological information BI as a pre-bathing pulse rate.

In the same manner as before bathing, the biological sensor 20 measures the blood oxygen saturation level and the pulse rate of the subject every several tens of second as the biological information BI. The biological sensor 20 transmits the biological information BI to the portable terminal 40 whenever the biological information BI is measured. Then, the portable terminal 40 transmits the biological information BI to the server 50 through the second communication unit whenever the biological information BI is received.

An example will now be given in which the subject ends bathing at a certain point of time. That is, the subject leaves the bathroom. The bathing detection sensor 30 detects that the subject finished bathing and transmits a non-detection signal through the communication unit to the portable terminal 40.

Then, the portable terminal 40 transmits the non-detection signal through the second communication unit to the server 50. Upon acquisition of the non-detection signal from the biological sensor 20, the server 50 executes the bathing determination process. In other words, upon acquisition of the non-detection signal, the server 50 determines that the subject finished bathing.

When determining that the subject finished bathing, the server 50 executes the first acquisition process. More specifically, the server 50 acquires the blood oxygen saturation level taken during the first measurement period that is at least part of the bathing period. More specifically, the server 50 extracts the blood oxygen saturation level from the biological information BI taken during the first measurement period. The server 50 acquires the minimum value of the extracted blood oxygen saturation levels as the first oxygen saturation level.

In the same manner as during bathing, the biological sensor 20 measures the blood oxygen saturation level and the pulse rate of the subject every several tens of second as the biological information BI. The biological sensor 20 transmits the biological information BI to the portable terminal 40 whenever the biological information BI is measured. Then, the portable terminal 40 transmits the biological information BI to the server 50 through the second communication unit whenever the biological information BI is received.

When 30 minutes elapses after the subject finishes bathing, the server 50 executes the second acquisition process and the pulse rate data acquisition process. More specifically, the server 50 acquires the blood oxygen saturation level and the pulse rate of the subject taken during the second measurement period, which is at least part of the period from when determined that the subject finished bathing to when determined that the subject started bathing. As described above, the second measurement period is a period from when determined that the subject finished bathing to when the power of the biological sensor 20 is turned off. Thus, as long as the power of the biological sensor 20 is not turned off within 30 minutes, the second measurement period includes 30 minutes that is a time point after a predetermined specific time from when determined that the subject finished bathing in the bathing determination process. More specifically, in the second acquisition process, the server 50 extracts the biological information BI taken at the time point closest to 30 minutes after determining that the subject finished bathing from the biological information BI taken during the second measurement period. Then, in the second acquisition process, the server 50 acquires the blood oxygen saturation level included in the extracted biological information BI as the second oxygen saturation level.

Further, the server 50 acquires the pulse rate after a predetermined specified time elapses from when determining in the bathing determination process that the subject started bathing as a post-bathing pulse rate. The specified time is 10 minutes. The specified time includes the second measurement period. More specifically, in the pulse rate data acquisition process, the server 50 extracts the biological information BI taken at the time point closest to 10 minutes after determining that the subject finished bathing from the biological information BI taken during the second measurement period. Then, in the pulse rate data acquisition process, the server 50 acquires the pulse rate included in the extracted biological information BI as the post-bathing pulse rate.

The server 50 executes the determination process. The server 50 determines whether a value obtained by subtracting the first oxygen saturation level from the second oxygen saturation level is less than or equal to a predetermined value set within a range of 3% to 4%, inclusive. The predetermined value is, for example, 3.5%. That is, the server 50 determines whether the first condition is satisfied.

Further, the server 50 determines whether the post-bathing pulse rate is greater than the pre-bathing pulse rate by a predetermined reference value or greater. That is, the server 50 determines whether the second condition is satisfied.

In the determination process, the server 50 determines that the heart failure-related health condition of the subject is deteriorating when the first condition and the second condition are both satisfied. Further, in the determination process, the server 50 determines that the heart failure-related health condition of the subject is not deteriorating when one of the first condition and the second condition is not satisfied or when both of the first condition and the second condition are not satisfied.

The server 50 executes the output process. In the output process, the server 50 outputs the determination result of the determination process to the portable terminal 40. For example, when the determination result is “the heart failure-related health condition of the subject is deteriorating,” the server 50 transmits a first signal to the portable terminal 40. Upon receipt of the first signal, the portable terminal 40 can be configured to display a text, image, or the like (e.g., on a display device) indicating that the heart failure-related health condition of the subject is deteriorating on the display of the portable terminal 40. Further, upon receipt of the first signal, the portable terminal 40 may show text, image, or the like prompting the subject to visit a hospital in addition to the text, image, or the like indicating that the cardiac arrest-related health condition of the subject is deteriorating on the display of the portable terminal 40. For example, when the determination result is “the heart failure-related health condition of the subject is not deteriorating,” the server 50 transmits a second signal to the portable terminal 40. Upon receipt of the second signal, the portable terminal 40 is configured to display a text, image, or the like (e.g., on a display device) indicating that the heart failure-related health condition of the subject is not deteriorating on the display of the portable terminal 40. In the output process, the server 50 also outputs the determination result to the external terminal 60. In the same manner as the portable terminal 40, the external terminal 60 outputs the determination result to the display of the external terminal 60 or a display connected to the external terminal 60.

### Technical Advantages of Second Exemplary Embodiment

(2-1) In the second embodiment, in the determination process, when the post-bathing pulse rate is greater than the pre-bathing pulse rate by the predetermined reference value or greater, the server 50 determines that a heart failure-related health condition of the subject is deteriorating. As described above, with a heart failure-related patient having a history of rehospitalization and a heart failure-related patient having no history of rehospitalization, when the average value of the pulse rate before bathing is used as a reference, the pulse rate taken 10 minutes after bathing is greater than the reference. The configuration described above allows for determinations to be given taking into consideration the effect of bathing.

(2-2) In the second embodiment, in the determination process, when a value obtained by subtracting the first oxygen saturation level from the second oxygen saturation level is less than or equal to a predetermined value set within a range of 3% to 4%, inclusive, the server 50 determines that the heart failure-related health condition of the subject is deteriorating Such determination allows for further accurate evaluation of deterioration in the heart failure-related health condition caused by bathing.

A third embodiment of a healthcare management system will now be described with reference to the drawings.

### Third Exemplary Embodiment

### Overall Configuration

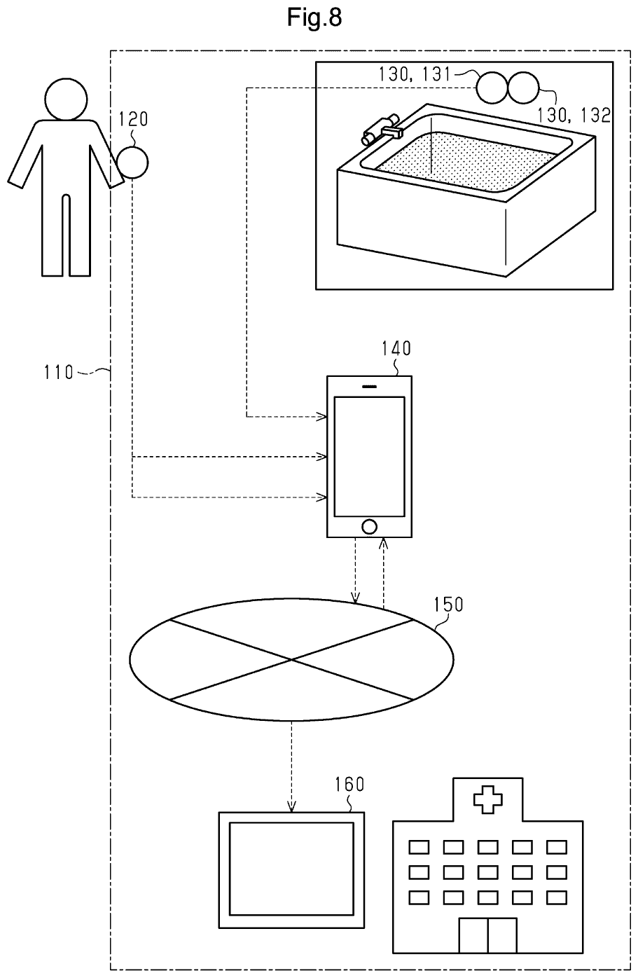

As shown in FIG. 8, a healthcare management system 110 includes a biological sensor 120, bathing detection sensors 130, a portable terminal 140, a server 150, and an external terminal 160.

The biological sensor 120 includes the functionality of a pulse oximeter, the functionality of a temperature sensor, and the functionality of an acceleration sensor. The biological sensor 120 is a wearable terminal attachable to a wrist or finger of a subject. The biological sensor 120 acquires four types of biological information BI.

The biological sensor 120 can be configured to implement the functionality of a pulse oximeter to measure the blood oxygen saturation level and pulse rate of a subject as the biological information BI. Preferably, the biometric sensor 120 calculates the blood oxygen saturation level and the pulse rate from transmittance of red light and infrared light when irradiating the subject with the red light and the infrared light. The biological sensor 120 implements the functionality of a temperature sensor to measure the body temperature of the subject as the biological information BI. The biological sensor 120 implements the functionality of an acceleration sensor to measure the acceleration of the subject as the biological information BI. The acceleration of the subject may be used to detect movement of the subject such as the number of steps the subject walks.

Although not shown in the drawings, the biological sensor 120 includes a storage unit and a communication unit. The storage unit and the communication unit may be configured by a single chip or module or by independent chips or modules. The storage unit includes a ROM, which is capable of only reading data, a non-volatile memory, which is capable of reading and writing data, a volatile memory, which is capable of reading and writing data, and the like. The same applies to the following.

The storage unit stores the detected biological information BI. The communication unit is configured to communicate with an external device. The communication unit uses, for example, Bluetooth® for communication. The biological sensor 120 is configured to transmit the biological information BI, which is stored in the storage unit, through the communication unit to the external device.

The server 120 is configured to execute an acquisition process. The acquisition process acquires the biological information BI over time. As described above, the biological sensor 120 is configured to measure the blood oxygen saturation level, the pulse rate, the body temperature, and the acceleration of the subject as the biological information BI. The biological sensor 120 stores the measured biological information BI in the storage unit. The biological sensor 120 transmits the biological information BI to the portable terminal 140 whenever the biological information BI is measured.

The bathing detection sensors 130 include a human presence sensor 131 and a flow sensor 132. The human presence sensor 131 is set (e.g., positioned) in the bathroom. The human presence sensor 131 is configured to detect the presence of a person in the bathroom. That is, the human presence sensor 131 is configured to detect the presence of the subject in the bathroom. The human presence sensor 131 is, for example, a pyroelectric infrared sensor that detects a change in the temperature of a heat source within a detection area. Further, the human presence sensor 131 may be incorporated in a light bulb or the like in the bathroom. Although not shown in the drawings, the human presence sensor 131 includes a communication unit. The communication unit is configured to communicate with an external device. The communication unit uses, for example, Bluetooth® for communication.

When detecting a person in the bathroom, the human presence sensor 131 transmits a detection signal to the portable terminal 140. When no person is detected in the bathroom, the human presence sensor 131 transmits a non-detection signal to the portable terminal 140. The human presence sensor 131 is configured to transmit the detection signal and the non-detection signal to the portable terminal 140 through the communication unit.

The flow sensor 132 is set (e.g., positioned) in a shower head in the bathroom. The flow sensor 132 is configured to detect the presence of a person in the bathroom when detecting a flow rate that is greater than or equal to a predetermined specified flow rate. The specified flowrate is set based on the flow rate of water in the shower head when bathing (showering) that was checked through experiments or the like. Although not shown in the drawings, the human presence sensor 131 includes a communication unit. The communication unit is configured to communicate with an external device. The communication unit uses, for example, Bluetooth® for communication.

When detecting a person in the bathroom, the flow sensor 132 transmits a detection signal to the portable terminal 140. When no person is detected in the bathroom, the flow sensor 132 transmits a non-detection signal to the portable terminal 140. The flow sensor 132 is configured to transmit the detection signal and the non-detection signal to the portable terminal 140 through the communication unit.

In the third embodiment, the portable terminal 140 is a smartphone. Although not shown in the drawings, the portable terminal 140 includes a storage unit, a first communication unit, and a second communication unit. In the third embodiment, the first communication unit uses Bluetooth® for communication. The second communication unit uses a portable telephone communication network for communication. Accordingly, in the portable terminal 140, the second communication unit has a wider communication range than the first communication range.

The portable terminal 140 is configured to acquire the biological information BI from the biological sensor 120 through the first communication unit. Further, the portable terminal 140 is configured to acquire the detection signal and the non-detection signal from the bathing detection sensor 130 through the first communication unit. The portable terminal 140 is configured to store the biological information BI, the detection signal, and the non-detection signal in the storage unit. The portable terminal 140 is configured to transmit the biological information BI, the detection signal, and the non-detection signal, which are stored in the storage unit of the portable terminal 140, to the server 150 through the second communication unit.

Although not shown in the drawings, the server 150 includes a control unit, a storage unit, and a communication unit. More specifically, the control unit is a CPU. The communication unit uses the portable telephone communication network for communication. The server 150 performs various types of processes by executing programs stored in the storage. Further, the server 150 is configured to execute a communication process to transmit a signal to the portable terminal 140.

The server 150 is configured to execute a bathing determination process. Specifically, the bathing determination process determines when a subject starts bathing and finishes bathing. The server 150 is configured to execute the bathing determination process based on the determination of the two bathing detection sensors 130. More specifically, the server 150 acquires detection signals from both the human presence sensor 131 and the flow sensor 132 to determine whether the subject is taking a bath. Further, after determining that the subject is taking a bath, the server 150 acquires non-detection signals from both the human presence sensor 131 and the flow sensor 132 to determine whether the subject finished taking a bath. In this manner, the server 150 is configured to execute the bathing determination process based on the detections of the human presence sensor 131 and the flow sensor 132. The expression of “the server 150 is configured to execute” indicates that the control unit of the server 150 is configured to execute. The same applies to the following description.

The server 150 is configured to have the biological sensor 120 execute the acquisition process. As described above, the acquisition process acquires the biological information BI of the subject over time. The server 150 is configured to have the biological sensor 120 execute the acquisition process in one of a first measurement mode, a second measurement mode, and a third measurement mode. The measurement modes will be described later.

During a pre-bathing period, which is before determined that the subject started bathing in the bathing determination process, that is, in a state before the subject takes a bath, the server 150 has the biological sensor 120 acquire the biological information BI in the first measurement mode. More specifically, after a predetermined period elapses from when determining that the subject finished bathing in the bathing determination process, the server 150 resets the determination. After resetting the determination, the server 150 sets the period from when the determination is reset to when next determining that the subject started bathing as the pre-bathing period. The predetermined period may be set as, for example, a range within several tens of minutes to several hours. Further, when resetting the determination as described above, the server 150 transmits a first signal to the portable terminal 140. The first signal is transmitted to the portable terminal 140. Upon receipt of the first signal, the portable terminal 140 transmits a first mode signal to the biological sensor 120. Then, the biological sensor 120 starts measurement in the first measurement mode in the acquisition process. After the power of the biological sensor 120 is turned on, the biological sensor 120 performs measurement in the first measurement mode as long as no signal is received from the portable terminal 140. In this manner, the biological sensor 120 is in an initial state in the first measurement mode.

During a bathing period, which is from when determined that the subject started bathing to when determined that the subject finished bathing in the bathing determination process, that is, in a state in which the subject is bathing, the server 150 has the biological sensor 120 acquire the biological information BI in the second measurement mode. More specifically, when determining that the subject started bathing in the bathing determination process, the server 150 transmits a second signal to the portable terminal 140. Upon receipt of the second signal, the portable terminal 140 transmits a second mode signal to the biological sensor 120. Then, the biological sensor 120 starts measurement in the second measurement mode in the acquisition process.

During a post-bathing period, which is after determining that the subject finished bathing in the bathing determination process, that is, in a state after the subject finishes bathing, the server 150 has the biological sensor 120 acquire the biological information BI in the third measurement mode. More specifically, when determining that the subject finished bathing in the bathing determination process, the server 150 transmits a third signal to the portable terminal 140. Upon receipt of the third signal, the portable terminal 140 transmits a third mode signal to the biological sensor 120. Then, the biological sensor 120 starts measurement in the third measurement mode in the acquisition process. As described above, after a predetermined period elapses from when determining that the subject finished bathing, the server 150 resets the determination. Thus, the post-bathing period is the predetermined period from when determined that the subject finished bathing in the bathing determination process.

The server 150 is configured to execute a determination process. The server 150 determines whether there is an abnormality in the health condition of the subject based on a transition in the biological information BI acquired in the acquisition process. An abnormality in the health condition of the subject is determined when, for example, the two conditions described below are both satisfied.

According to an exemplary aspect, the minimum value of the blood oxygen saturation levels acquired in the second measurement mode and the third measurement mode is less than the average value of the blood oxygen saturation levels acquired in the first measurement mode by a predetermined threshold or greater.

According to an exemplary aspect, the maximum value of the pulse rates acquired in the second measurement mode and the third measurement mode is greater than the average value of the pulse rates acquired in the first measurement mode by a predetermined threshold or greater.

The server 150 is configured to execute an output process. The output process outputs the determination result of the determination process. More specifically, in the output process, the server 150 outputs the determination result of the determination process to the portable terminal 140, which is possessed by the subject, and the external terminal 160.

In the third embodiment, the external terminal 160 is a person computer (PC) set in a hospital. Although not shown in the drawings, the external terminal 160 includes a communication unit. In the third embodiment, the communication unit uses the portable phone communication network for communication. The external terminal 160 is configured to receive a signal from the server 150 through the communication unit.

### Measurement Modes

As described above, the biological sensor 120 executes the acquisition process in one of the first measurement mode, the second measurement mode, and the third measurement mode. The types of the biological information BI acquired in the first measurement mode, the second measurement mode, and the third measurement mode include the blood oxygen saturation level and the pulse rate.

In the first measurement mode, the biological sensor 120 measures the blood oxygen saturation level and the pulse rate every five minutes as the biological information BI. The biological sensor 120 stores the measured blood oxygen saturation level and pulse rate in the storage unit. The biological sensor 120 transmits the blood oxygen saturation level and the pulse rate whenever measured to the portable terminal 140. The portable terminal 140 transmits the blood oxygen saturation level and the pulse rate to the server 150.

In the first measurement mode, the biological sensor 120 also measures the body temperature every five minutes as the biological information BI. The biological sensor 120 stores the measured body temperature in the storage unit. The biological sensor 120 transmits the body temperature whenever measured to the portable terminal 140. The portable terminal 140 transmits the body temperature to the server 150.

Further, in the first measurement mode, the biological sensor 120 measures the acceleration of the subject as the biological information BI every second. The biological sensor 120 stores the measured acceleration in the storage unit. The biological sensor 120 transmits the acceleration whenever measured to the portable terminal 140. The portable terminal 140 transmits the acceleration to the server 150.

In the second measurement mode, the biological sensor 120 measures the blood oxygen saturation level and the pulse rate every second as the biological information BI. The biological sensor 120 stores the measured blood oxygen saturation level and pulse rate in the storage unit. The biological sensor 120 transmits the blood oxygen saturation level and the pulse rate whenever measured to the portable terminal 140. The portable terminal 140 transmits the blood oxygen saturation level and the pulse rate to the server 150.

In the second measurement mode, the biological sensor 120 stops measuring the body temperature of the subject. Further, in the second measurement mode, the biological sensor 120 stops measuring the acceleration of the subject. That is, in the second measurement mode, the body temperature and the acceleration of the subject are not measured.

In this manner, the first measurement mode differs from the second measurement mode in the type of the biological information BI acquired. More specifically, the biological information BI acquired in the first measurement mode includes the body temperature, and the biological information BI acquired in the second measurement mode does not include the body temperature. Further, the biological information BI acquired in the first measurement mode includes the acceleration, and the biological information BI acquired in the second measurement mode does not include the acceleration

Further, the first measurement mode differs from the second measurement mode in the measurement frequency of the biological information BI. More specifically, in the second measurement mode, the blood oxygen saturation level and the pulse rate are measured at a higher frequency than the first measurement mode.

In the third embodiment, the measurement frequency of the biological information BI and the type of the acquired biological information BI in the third measurement mode are both the same as the second measurement mode. In other words, the third measurement mode differs from the first measurement mode in the measurement frequency of the biological information BI and the type of the acquired biological information BI. The measurement frequency of the blood oxygen saturation level and the pulse rate in the third measurement mode is higher than the first measurement mode.

### Processing Performed by Healthcare Management System

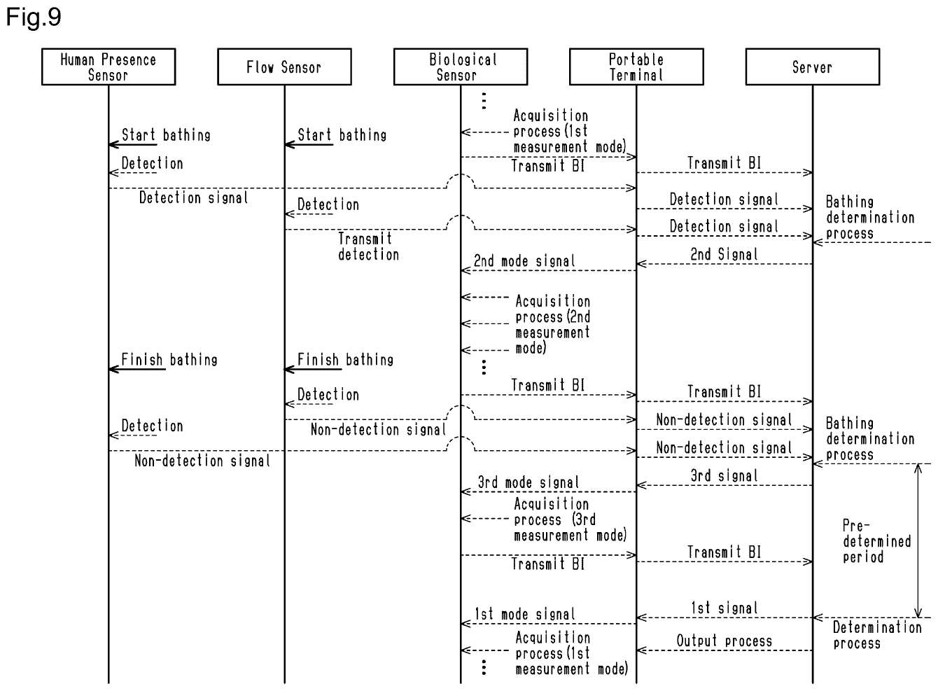

With reference to FIG. 9, one example of the processing performed by the healthcare management system 110 will now be described.

The power of the biological sensor 120 attached to the subject is first turned on. When the power is turned on, the biological sensor 120 performs measurements in the first measurement mode. The biological sensor 120 transmits the biological information BI to the portable terminal 140 whenever the biological information BI is measured. FIG. 9 does not show part of the biological information BI transmitted from the biological sensor 120 to the portable terminal 140.

Then, the portable terminal 140 transmits the biological information BI to the server 150 through the second communication unit whenever the biological information BI is received. The server 150 stores the biological information BI. FIG. 9 does not show part of the biological information BI transmitted from the portable terminal 140 to the server 150.

An example will now be described in which a subject starts bathing at a certain point of time. That is, the subject enters the bathroom. The human presence sensor 131 detects that the subject started bathing and transmits a detection signal through the communication unit to the portable terminal 140. Then, the portable terminal 140 transmits the detection signal received from the human presence sensor 131 to the server 150 through the second communication unit.

The flow sensor 132 detects that the subject started bathing and transmits a detection signal through the communication unit to the portable terminal 140. Then, the portable terminal 140 transmits the detection signal received from the flow sensor 132 to the server 150. through the second communication unit

Upon acquisition of the detection signals from the bathing detection sensors 130, the server 150 executes the bathing determination process. In other words, upon acquisition of the detection signal, the server 150 determines that the subject started bathing.

When determining that the subject started bathing, the server 150 transmits a second signal to the portable terminal 140. Upon receipt of the second signal, the portable terminal 140 transmits a second mode signal to the biological sensor 120. The biological sensor 120 receives the second mode signal.

After the subject starts bathing, the biological sensor 120 starts measurement in the second measurement mode in response to the second mode signal. The biological sensor 120 transmits the biological information BI to the portable terminal 140 whenever the biological information BI is measured. Then, the portable terminal 140 transmits the biological information BI to the server 150 through the second communication unit whenever the biological information BI is received. The server 150 stores the biological information BI.

An example will now be given in which the subject ends bathing at a certain point of time. That is, the subject leaves the bathroom. The flow sensor 132 detects that the subject finished bathing and transmits a non-detection signal to the portable terminal 140 through the communication unit. Then, the portable terminal 140 transmits the non-detection signal received from the flow sensor 132 to the server 150 through the second communication unit.

Further, the human presence sensor 131 detects that the subject finished bathing and transmits a non-detection signal through the communication unit to the portable terminal 140. Then, the portable terminal 140 transmits the non-detection signal received from the human presence sensor 131 to the server 150 through the second communication unit.

Upon acquisition of the detection signals from the bathing detection sensors 130, the server 150 executes the bathing determination process. In other words, upon acquisition of the detection signals, the server 150 determines that the subject finished bathing.

When determining that the subject finished bathing, the server 150 transmits a third signal to the portable terminal 140. Upon receipt of the third signal, the portable terminal 140 transmits a third mode signal to the biological sensor 120. The biological sensor 120 receives the third mode signal.

The biological sensor 120 starts measurement in the third measurement mode in response to the third mode signal. The biological sensor 120 transmits the biological information BI to the portable terminal 140 whenever the biological information BI is measured. Then, the portable terminal 140 transmits the biological information BI to the server 150 through the second communication unit whenever the biological information BI is received. The server 150 stores the biological information BI.

After a predetermined period elapses from when determining that the subject finished bathing, the server 150 resets the determination. Further, when resetting the determination, the server 150 transmits a first signal to the portable terminal 140. Upon receipt of the first signal, the portable terminal 140 transmits a first mode signal to the biological sensor 120. The biological sensor 120 receives the first mode signal. The biological sensor 120 starts measurement in the first measurement mode in response to the first mode signal. In this manner, when the predetermined period elapses after the subject finishes bathing, the biological sensor 120 returns to the initial state, in which the measurement mode is the first measurement mode.

Further, when resetting the determination, the server 150 executes the determination process. The server 150 determines whether there is an abnormality in the health condition of the subject based on a transition in the biological information BI acquired in the acquisition process. In the third embodiment, the server 150 determines whether there is an abnormality in the biological information BI from the blood oxygen saturation level and the pulse rate acquired in each measurement mode. The conditions for determining that there is an abnormality in the biological information BI are as described above.

Upon determination in the determination process whether there is an abnormality in the biological information BI, the server 150 is configured to execute the output process. The output process outputs the determination result of the determination process. More specifically, in the output process, the server 150 outputs the determination result of the determination process to the portable terminal 140, which is possessed by the subject, and the external terminal 160.

### Technical Advantages of Third Exemplary Embodiment

(3-1) In the third embodiment, the biological sensor 120 acquires the biological information BI in the first measurement mode during the pre-bathing period. Further, the biological sensor 120 acquires the biological information BI in the second measurement mode during the bathing period. The first measurement mode differs from the second measurement mode in the measurement frequency of the biological information BI and the type of the acquired biological information BI. With this configuration, the acquisition process is executed under conditions preferable for acquiring the biological information BI of the subject when the subject is taking a bath, which is a special environment. Accordingly, the above configuration avoids a situation in which the biological information BI is in excess or insufficient.

(3-2) In the third embodiment, the measurement frequency of the biological information BI is higher in the second measurement mode than in the first measurement mode. With this configuration, the biological information BI is acquired more often when the subject is bathing than before the subject bathes. This allows changes in the biological information BI when the subject is bathing to be detected with high accuracy.

(3-3) In the post-bathing period, the subject is not bathing. However, the biological information BI will tend to change due to the effect of bathing. In the third embodiment, when in the post-bathing period, the biological sensor 120 acquires the biological information BI in the third measurement mode. In the above configuration, the third measurement mode differs from the first measurement mode in the measurement frequency of the biological information BI and the type of the acquired biological information BI. With this configuration, the acquisition process is executed under conditions preferable for acquiring the biological information BI of the subject after the subject takes a bath, which is a special environment. Accordingly, the above configuration avoids a situation in which the biological information BI is in excess or insufficient.

(3-4) In the third embodiment, the measurement frequency of the biological information BI is higher in the third measurement mode than in the first measurement mode. With this configuration, the biological information BI is acquired more often after the subject bathes than before the subject bathes. This allows changes in the biological information BI after the subject bathes to be detected with high accuracy.

(3-5) In a post-bathing period, which is after the determination that the subject finished bathing in the bathing determination process, the biological information BI will tend to change due to the effect of bathing. In the third embodiment, the measurement frequency of the biological information BI and the type of the acquired biological information BI are the same in the second measurement mode and the third measurement mode. In this manner, the biological information BI is acquired in the post-bathing period in a manner similar to the bathing period.

(3-6) When the subject is bathing, the biological sensor 120 may not be able to detect the body temperature correctly due to the effect of hot water in the bath tub or hot water from the shower. Thus, the reliability of the body temperature measured by the biological sensor 120 is low when the subject is bathing, and the body temperature cannot be used as a parameter for determining whether there is an abnormality in the biological information BI. In the third embodiment, the biological information BI acquired in the second measurement mode does not include body temperature. This reduces the amount of data of the biological information BI that is transmitted and the amount of data that is stored in the server 150. Further, the reduction in the amount of transmitted data decreases battery consumption of the biological sensor 120.

(3-7) The acceleration of the subject is used to determine movement of the subject such as the number of steps the subject walks. Acceleration does not have to be detected when the subject is bathing, since the subject is not likely to walk or run. In the third embodiment, the biological information BI acquired in the second measurement mode does not include acceleration. With this configuration, unnecessary information is not acquired.

(3-8) When the subject has a history of heart failure, the blood oxygen saturation level and the pulse rate tend to differ between when the subject is bathing and when the subject is not bathing. In the third embodiment, the type of the biological information BI acquired in the first measurement mode, the second measurement mode, and the third measurement mode includes at least one of the blood oxygen saturation level and the pulse rate. Accordingly, as long as the biological information BI includes at least one of the blood oxygen saturation level and the pulse rate, the determination process will readily determine an abnormality in the subject, more specifically, an abnormality in the heart failure-related health condition.

(3-9) In the third embodiment, the server 150 executes the bathing determination process based on the detections of the human presence sensor, which is set (e.g., positioned) in the bathroom and configured to detect the subject, and the flow sensor, which is set (e.g., positioned) in a shower head in the bathroom. The detections of the two bathing detection sensors 130 allows for determination that the subject started bathing and finished bathing. This avoids erroneous determination of when the subject started or finished bathing.

(3-10) In the third embodiment, the contents are the same in the second measurement mode and the third measurement mode. When determining that the subject finished bathing, the server 150 transmits the third signal. This allows the portable terminal 140 and the biological sensor 120 to recognize the post-bathing period. As long as the post-bathing period can be recognized, for example, the biological sensor 120 and the portable terminal 140 can label the acquired biological information BI as data of the post-bathing period.

### Modified Examples of the First Embodiment and the Second Embodiment

It is noted that the first embodiment, the second embodiment, and the modified examples described below may be combined as long as there is no technical contradiction as would be appreciated to one skilled in the art.

### Modified Examples of Overall Configuration

In the first and second embodiments, the portable terminal 40 does not have to be possessed by the subject according to an exemplary aspect. Further, the determination result does not have to be output to a smartphone and may be output to any device that can perform communication. For example, a desk top personal computer or the like may be used instead of the portable terminal 40.

In the first and second embodiments, there is no limitation to how communication is performed by the biological sensor 20, the portable terminal 40, the server 50, and the external terminal 60 according to an exemplary aspect. For example, communication may be performed in accordance with the set of standards defined by IEEE 802.11 or the like. Further, communication is not limited to wireless communication. For example, the biological sensor 20 and the portable terminal 40 may be wire-connected in a manner allowing for communication. Further, the biological sensor 20 does not need to have the functionality for communicating with the portable terminal 40. In this case, for example, the biological sensor 20 is configured to communicate with a cradle corresponding to the biological sensor 20, and the cradle is configured to communicate with the portable terminal 40 through wire connection. In this manner, if the biological sensor 20 has no communication unit for communicating with the portable terminal 40, this allows the biological sensor 20 to be more compact.

In the first and second embodiments, the biological sensor 20 is not limited to a pulse oximeter according to an exemplary aspect. Instead, the biological sensor 20 may be any sensor that measures the blood oxygen saturation level and the pulse rate. Further, in addition to the blood oxygen saturation level and the pulse rate, the biological sensor 20 may be a sensor that detects other parameters such as the body temperature and the respiration rate. Further, the healthcare management system 10 may include a plurality of biological sensors 20. In this case, the blood oxygen saturation level and the pulse rate may be measured by different biological sensors 20. In the first and second embodiments, “pulse rate” indicates the number of pulsations counted each time the heart contracts and includes the heart rate, which is the number of heartbeats.

In the first and second embodiments, the biological sensor 20 may start and stop measurement of the biological information BI at time points other than when the power is turned on and off according to an exemplary aspect. For example, the server 50 may transmit a signal for starting measurement through the portable terminal 40 so that the biological sensor 20 starts measuring the biological information BI. Further, for example, when determined in the bathing determination process that the subject started bathing, the biological sensor 20 may start measuring the biological information BI.

In the first and second embodiments, there is no limitation to the measurement interval of the biological sensor 20 according to an exemplary aspect. The biological sensor 20 may acquire the biological information BI at any time point. Further, the biological sensor 20 may perform a measurement at least once in the first measurement period and at least once in the second measurement period. Further, for example, the server 50 may store the data of the blood oxygen saturation level in the second measurement period as the second oxygen saturation level in order to compare the second oxygen saturation level with the first oxygen saturation level measured on another day. That is, the second acquisition process and the first acquisition process do not have to be executed on the same day.

In the first and second embodiments, the biological sensor 20 does not have to transmit the biological information BI to the portable terminal 40 whenever the biological information BI is measured according to an exemplary aspect. Segments of the biological information BI may be stored in the biological sensor 20 and transmitted at predetermined intervals. The same applies to the portable terminal 40. The portable terminal 40 may collectively transmit the segments of the biological information BI to the server 50.

In the first and second embodiments, the healthcare management system 10 does not have to include the biological sensor 20, the portable terminal 40, the server 50, and the external terminal 60 according to an exemplary aspect. The healthcare management system 10 only needs to include an element or elements configured to execute the bathing determination process, the first acquisition process, the second acquisition process, the pulse rate data acquisition process, the determination process, and the output process. These processes may be executed by the same element or by different elements. Further, in the first embodiment, the biological sensor 20 may execute the bathing determination process. The portable terminal 40 may execute the first acquisition process, the second acquisition process, and the pulse rate data acquisition process. Further, the server 50 may execute the determination process and the output process.

### Modified Examples of Bathing Detection Sensor and Bathing Determination Process

In the first and second embodiments, the bathing detection sensor 30 may be changed as long as it can detect when a subject is bathing according to the exemplary aspects.

In an exemplary aspect, the bathing detection sensor 30 may be a door sensor attached to a bathroom door and configured to detect whether the bathroom door is in an open state or a closed state. In this case, for example, in the bathing determination process, the server 50 determines that the subject started bathing when the door sensor detects that the bathroom door switched from an open state to a closed state. Further, in the bathing determination process, after determining that bathing started, the server 50 determines that the subject finished bathing when the door sensor first detects that the bathroom door is in an open state.

In an exemplary aspect, the bathing detection sensor 30 may be a temperature sensor and a humidity sensor that are set (e.g., positioned) in the bathroom. In this case, for example, in the bathing determination process, the server 50 determines that the subject started bathing when a positive change amount of the temperature detected by the temperature sensor is greater than or equal to a predetermined first temperature change amount and a positive change amount of the humidity detected by the humidity sensor is greater than or equal to a predetermined first humidity change amount. Further, in the bathing determination process, the server 50 determines that the subject finished bathing when a negative change amount of the temperature detected by the temperature sensor is greater than or equal to a predetermined second temperature change amount and a negative change amount of the humidity detected by the humidity sensor is greater than or equal to a predetermined second humidity change amount. The first temperature change amount, the second temperature change amount, the first humidity change amount, and the second humidity change amount may be set through experiments or the like based on measurements of each change amount taken when bathing is started and when bathing is finished.

Further, in an exemplary aspect, the bathing detection sensor 30 may be an acceleration sensor attached to a subject. For example, the acceleration sensor may be incorporated in a wearable device such as a smart watch or a smart band. In this case, for example, in the bathing determination process, the server 50 determines when the subject started bathing and when the subject finished bathing by detecting the posture of the subject from the detection value of the acceleration sensor and obtaining the similarity of the posture to a predetermined bathing posture.

In an exemplary aspect, the bathing detection sensor 30 may be a flow sensor set (e.g., positioned) in a shower head. In this case, in the bathing determination process, the server 50 determines that the subject started bathing when the flow sensor detects a flow rate that is greater than or equal to a predetermined specified flow rate. Further, in the bathing determination process, the server 50 determines that the subject finished bathing when the flow sensor detects a flow rate that is less than the predetermined specified flow rate. The specified flow rate is obtained through experiments or the like.

In an exemplary aspect, the bathing detection sensor 30 may be a microphone sent in the bathroom. In this case, in the bathing determination process, the server 50 determines that the subject started bathing when the microphone detects the sound of shower. Further, in the bathing determination process, after the sound of shower is detected, the server 50 determines that the subject finished bathing when the microphone does not detect the sound of shower for a predetermined time. The sound of shower is prerecorded and analyzed so that the sound of shower can be detected based on the analysis. When the microphone detects a certain volume of sound or greater, the server 50 may determine that the sound of shower is detected.

In an exemplary aspect, the bathing determination process may be executed with an element other than the bathing detection sensor 30. For example, the bathing determination process may be executed based on the measurements of a water meter and an electricity meter or based on the usage situation of a water heater. For example, when starting bathing, the subject may input a signal indicating that bathing started to the portable terminal 40. Further, when finishing bathing, the subject may input a signal indicating that bathing finished to the portable terminal 40. The portable terminal 40 may transmit these signals to the server 50, and the server 50 may execute the bathing determination process based on the signals. As described above, the healthcare management system 10 does not necessarily have to include the bathing detection sensor 30.

In the first and second embodiments, the human presence sensor, which is the bathing detection sensor 30, may be set (e.g., positioned) in a changing room from where the subject enters the bathroom. In this case, in the same manner as the above embodiment, in the bathing determination process, the server 50 determines that the subject started bathing when the human presence sensor no longer detects a person. In the bathing determination process, after determining that the subject started bathing, the server 50 determines that the subject finished bathing when the human presence sensor detects a person.

The bathing determination process may be executed by combining the first embodiment, the second embodiment, and the bathing determination methods described above. That is, the bathing determination process may determine that the subject started bathing when such a determination is obtained in a number of methods.

For a period of several tens of minutes after bathing, the blood oxygen saturation level and the pulse rate are affected by bathing and tend to change in the same manner as during bathing. Thus, for example, the server 50 may determine that the subject finished bathing several tens of minutes after receiving a non-detection signal transmitted from the bathing detection sensor 30. That is, the subject may be determined to be bathing for a period of several tens of minutes after actually finishing bathing. In this case, after the specific time will refers to the time point at which the specific time elapses after determined in the bathing determination process that the subject finished bathing.

### Modified Examples of First Acquisition Process

In the first embodiment, there is no limitation to the first oxygen saturation level acquired in the first acquisition process as long as it is a value based on the blood oxygen saturation level of the subject measured in the first measurement period. For example, the first oxygen saturation level may be the maximum value, the minimum value, or the like of the blood oxygen saturation level during the first measurement period. Further, the first oxygen saturation level may be an instantaneous value taken at a specific time point during the first measurement period. The first oxygen saturation level may be the most recent value of the blood oxygen saturation level acquired during the first measurement period. The first oxygen saturation level may be a value obtained by performing statistical processing other than averaging on the blood oxygen saturation level. The same applies to the second embodiment.

In the first and second embodiments, there is no limitation to when the first acquisition process is executed. For example, the server 50 may store the biological information BI transmitted from the portable terminal 40 and calculate the average value of the blood oxygen saturation level during the first measurement period at a predetermined time to execute the first acquisition process. Further, when the first oxygen saturation level is the most recent value of the blood oxygen saturation level of the subject taken during the first measurement period, the first acquisition process may be executed whenever the biological information BI is received from the portable terminal 40.

In the first and second embodiments, the first measurement period is not limited to the entire bathing period from when determined that the subject started bathing to when determined that the subject finished bathing. The first measurement period may be only part of the bathing period from when determined that the subject started bathing to when determined that the subject finished bathing.

### Modified Examples of Second Acquisition Process

In the first embodiment, the second oxygen saturation level acquired in the second acquisition process is not limited to the average value of the blood oxygen saturation level of the subject in the second measurement period. The second oxygen saturation level may be the maximum value, the minimum value, or the like of the blood oxygen saturation level during the second measurement period. Further, the second oxygen saturation level may be an instantaneous value taken at a specific time point during the second measurement period. The second oxygen saturation level may be the most recent value of the blood oxygen saturation level acquired during the second measurement period. The second oxygen saturation level may be a value obtained by performing statistical processing other than averaging on the blood oxygen saturation level. The second oxygen saturation level may be a fixed value derived from the blood oxygen saturation level of the subject during the second measurement period. The same applies to the second embodiment.

In the first and second embodiments, the second measurement period may be the entire bathing period from when determined that the subject started bathing to when determined that the subject finished bathing. That is, the second measurement period may be the entire period excluding the first measurement period.

In the first and second embodiments, there is no limitation to when the second acquisition process is executed. For example, the server 50 may store the biological information BI transmitted from the portable terminal 40 and calculate the average value of the blood oxygen saturation level during the second measurement period at a predetermined time to execute the second acquisition process. Further, when the second oxygen saturation level is the most recent value of the blood oxygen saturation level of the subject taken during the second measurement period, the second acquisition process may be executed whenever the biological information BI is received from the portable terminal 40.

### Modified Examples of Pulse Rate Data Acquisition Process

In the first and second embodiments, the pulse rate data acquisition process may acquire the pulse rate in any of the period during which the subject is bathing and during which the subject is not bathing.

In the first embodiment, the first pulse rate acquired in the pulse rate data acquisition process is not limited to the average value of the pulse rate of the subject in the first measurement period. The first pulse rate may be the maximum value, the minimum value, or the like of the pulse rate in the first measurement period. Further, the first pulse rate may be an instantaneous value taken at a specific time point during the first measurement period. The first pulse rate may be the most recent value of the pulse rate acquired during the first measurement period. The first pulse rate may be a value obtained by performing statistical processing other than averaging on the pulse rate. The same applies to the second pulse rate. Further, the same applies to the pre-bathing pulse rate and the post-bathing pulse rate in the second embodiment.

In the first embodiment, there is no limitation to when the pulse rate data acquisition process is executed. For example, the server 50 may store the biological information BI transmitted from the portable terminal 40 and calculate the average value of the pulse rate at a predetermined time during the first measurement period as the first pulse rate to execute the pulse rate data acquisition process. Further, when a representative value of the first pulse rate or the like is the most recent value of the blood oxygen saturation level, the pulse rate data acquisition process is executed whenever the biological information BI is received from the portable terminal 40.

### Modified Example of Determination Process

In the first and second embodiments, there is no limitation to the determination process as long as a heart failure-related health condition of the subject is determined based on the first oxygen saturation level, the second oxygen saturation level, and a change in the pulse rate. That is, there is no limitation to the first and second conditions in the first and second embodiments. Further, the conditions related to the first oxygen saturation level and the second oxygen saturation level may be combined with the conditions related to changes in the pulse rate to execute the determination process.

The change in the pulse rate does not have to be obtained from a comparison of the first pulse rate and the second pulse rate or a comparison of the pre-bathing pulse rate and the post-bathing pulse rate. The change in the pulse rate may be obtained by comparing the pulse rates of any two time points. For example, the change in the pulse rate may be obtained from a comparison of the maximum value and the minimum value measured during bathing or the change amount of the pulse rate over any time.

In the first embodiment, there is no limitation to the first threshold value. The first threshold value may vary between different subjects or between the health condition and situation of the same subject. Accordingly, the first threshold value is preferably set for each subject or in accordance with the health condition and situation of the subject. The same applies to the second threshold value.

In the second embodiment, the specified time is not limited to 10 minutes.

In the second embodiment, the specific time is not limited to 30 minutes.

In the first embodiment, there is no limitation to when the determination process is executed. For example, the server 50 may execute the determination process at a predetermined time. In the first embodiment, when using the most recent values of the first oxygen saturation level and the first pulse rate, the determination process may be executed whenever the first acquisition process and the pulse rate data acquisition process are executed. This allows for determination of a heart failure-related health condition of the subject in real time.

When determining a heart failure-related health condition of the subject, the determination process may be executed based on only a first oxygen concentration and a second oxygen concentration. That is, in the determination process, determinations do not have to be performed based on a change in the pulse rate. Further, the pulse rate data acquisition process does not necessarily have to be performed.

### Modified Examples of the Output Process

In the first and second embodiments, the server 50 may execute the output process on only the portable terminal 40 or only the external terminal 60.

In the first and second embodiments, the server 50 may output the biological information BI together with the determination result.

In the first and second embodiments, there is no limitation to the output process as long as there is a device configured to output the determination result of the determination process. For example, the output destination may be a warning device that uses at least one of sound, vibration, and light to issue a warning. A warning using light includes an image or text shown on a display. There is no limitation to the owner of the output destination device. The output destination is not limited to a device having a warning functionality. Preferably, the server 50 allows for visual confirmation of the device to which data is output.

In the first and second embodiments, the portable terminal 40, which receives a signal in the output process, may issue a notification of the determination result using voice or vibration. Further, the portable terminal 40 may output the determination result through at least one of sound, light, and vibration.

In the first and second embodiments, there is no limitation to when the output process is executed. For example, the server 50 may store the determination result in the storage unit and execute the output process at a predetermined time point. In the same manner, the portable terminal 40, which receives the signal transmitted in the output process, may display the determination result at a predetermined time point.

### CLAUSES

A technical concept that can be recognized from the first embodiment, the second embodiment, and the modified examples will now be described.

A healthcare management system, configured to execute processes including: a bathing determination process for determining when the subject starts bathing and when the subject finishes bathing; a first acquisition process for acquiring a first oxygen saturation level that is a blood oxygen saturation level of the subject measured during a first measurement period, the first measurement period being at least part of a bathing period from when determined that the subject started bathing to when determined that the subject finished bathing in the bathing determination process; a second acquisition process for acquiring a second oxygen saturation level that is the blood oxygen saturation level of the subject measured during a second measurement period, the second measurement period being at least part of a non-bathing period from when determined that the subject finished bathing to when determined that the subject started bathing in the bathing determination process; a determination process for determining a heart failure-related health condition of the subject based on the first oxygen saturation level and the second oxygen saturation level; and an output process for outputting a determination result of the determination process.

### Modified Examples of Third Exemplary Embodiment

The third exemplary embodiment and the modified example described below may be combined as long as there is no technical contradiction.

Japanese Laid-Open Patent Publication No. 2022-164560 describes an information processing system including a biological information acquisition unit, an event information acquisition unit, an abnormality detection unit, and a notification unit. The biological information acquisition unit acquires biological information of the subject. The event information acquisition unit acquires event information as information related to an event that affects the biological information of a subject. The abnormality detection unit detects an abnormality in the biological information based on the biological information and the event information. The notification unit notifies the subject of information on the detected abnormality.

The subject from which the biological information is acquired performs various types of actions on a daily basis. The risk of an abnormality occurring in the biological information of the subject differs between each type of action. Thus, if the same biological information is acquired regardless of the action the subject is performing, the biological information may be in excess or be insufficient. Since the information processing system described in Japanese Patent Laid-Open Patent Publication No. 2022-164560 does not take this point into consideration, there is room for improvement.

According to the exemplary aspects described herein, the bathing determination process, the first acquisition process, the second acquisition process, the pulse rate data acquisition process, the determination process, and the output process of the first and second embodiments are not all required to avoid a situation in which the acquired biological information is in excess or insufficient. As long as the acquisition process, the bathing determination process, and the determination process can be executed in the healthcare management system 100, a situation in which the acquired biological information is in excess or insufficient may be avoided.

In the third embodiment, the portable terminal 140 does not have to be possessed by the subject. Further, the determination result does not have to be output to a smartphone and may be output to any device that can perform communication. For example, a desk top personal computer or the like may be used instead of the portable terminal 140.

In the third embodiment, there is no limitation to how communication is performed by the biological sensor 120, the bathing detection sensors 130, the portable terminal 140, the server 150, and the external terminal 160. For example, communication may be performed in accordance with the set of standards defined by IEEE 802.11 or the like. Further, communication is not limited to wireless communication. For example, the biological sensor 120 and the portable terminal 140 may be wire-connected in a manner allowing for communication. Further, the biological sensor 120 does not need to have the functionality for communicating with the portable terminal 140. In this case, for example, the biological sensor 120 is configured to communicate with a cradle corresponding to the biological sensor 120, and the cradle is configured to communicate with the portable terminal 140 through wire connection. In this manner, if the biological sensor 120 has no communication unit for communicating with the portable terminal 140, this allows the biological sensor 120 to be more compact.

In the third embodiment, the biological sensor 120 does not have to include the functionalities of a pulse oximeter, a temperature sensor, and an acceleration sensor. The biological sensor 120 may be configured to acquire at least one of the biological information BI of the subject. Further, the healthcare management system 110 may include multiple biological sensors 120, and the blood oxygen saturation level and the pulse rate may be measured by separate biological sensors 120. In the third embodiment, “pulse rate” indicates the number of pulsations counted each time the heart contracts and includes the heart rate, which is the number of heartbeats.

In the third embodiment, there is no limitation to the four types of biological information BI.

In the third embodiment, the biological sensor 120 may start and stop measurement of the biological information BI at time points other than when the power is turned on and off. For example, the server 150 may transmit a signal for starting measurement through the portable terminal 140 so that the biological sensor 120 starts measuring the biological information BI.

In the third embodiment, the biological sensor 120 does not have to transmit the biological information BI to the portable terminal 140 whenever the biological information BI is measured. Segments of the biological information BI may be stored in the biological sensor 120 and transmitted at predetermined intervals. The same applies to the portable terminal 140. The portable terminal 140 may collectively transmit the segments of the biological information BI to the server 150.

In the third embodiment, the healthcare management system 110 does not have to include the biological sensor 120, the portable terminal 140, the server 150, and the external terminal 160. Any element of the healthcare management system 110 may be configured to execute the acquisition process, the bathing determination process, the determination process, and the output process. These processes may be executed by the same element or by different elements. For example, in the third embodiment, the bathing detection sensors 130 may execute the bathing determination process, and the portable terminal 140 may execute the determination process and the output process.

In the third embodiment, as long as the determination process determines an abnormality in the subject based on transition of the biological information BI, there is no limitation to the determination method and the determination condition. The transition of the biological information BI may include the transition of at least one type of the biological information BI.

In the third embodiment, the output process does not have to be executed. For example, the output process may be executed by a device such as the portable terminal 140 only when there is an output request for the determination result.

In the third embodiment, for example, when starting bathing, the subject may input a signal indicating that bathing started to the portable terminal 140. Further, when finishing bathing, the subject may input a signal indicating that bathing finished to the portable terminal 140. The portable terminal 140 may transmit these signals to the server 150, and the server 150 may execute the bathing determination process in response to the signals. In this configuration, the healthcare management system 110 does not necessarily have to include the bathing detection sensors 130.

In the third embodiment, the bathing detection sensors 130 are not limited to the human presence sensor 131 and the flow sensor 132. For example, the bathing detection sensors 130 may include a door sensor attached to a bathroom door and configured to detect whether the bathroom door is in an open state or a closed state. In this case, for example, in the bathing determination process, the server 150 determines that the subject started bathing when the door sensor detects that the bathroom door switched from an open state to a closed state. Further, in the bathing determination process, after determining that bathing started, the server 150 determines that the subject finished bathing when the door sensor first detects that the bathroom door is in an open state.

Further, the bathing detection sensors 130 may include a human presence sensor set in a changing room from where the subject enters the bathroom and configured to detect the subject. In this case, in the bathing determination process, the server 150 determines that the subject started bathing when the human presence sensor no longer detects a person. In the bathing determination process, after determining that the subject started bathing, the server 150 determines that the subject finished bathing when the human presence sensor detects a person.

Further, the bathing detection sensors 130 may be an acceleration sensor attached to a subject. In this case, in the bathing determination process, the server 150 determines when the subject started bathing and when the subject finished bathing by detecting movement of the subject from the detection value of the acceleration sensor and obtaining the similarity of the movement to a predetermined bathing movement. The acceleration sensor may use the functionality of the acceleration sensor in the biological sensor 120.

As described above, the bathing determination process may be executed based on the detection of the door sensor set on the door of the bathroom and configured to detect an open state and a closed state of the door, the human presence sensor set in the bathroom or in the changing room and configured to detect the subject, and the flow sensor set in the shower head in the bathroom. The bathing determination process may be executed based on the detection of at least one of the above sensors. Instead of the above sensors, any sensor configured to detect bathing of the subject may be used as the bathing detection sensor 130. For example, the bathing determination process may be executed based on the detection result of the biological sensor 120 attached to the subject and configured to acquire the biological information BI of the subject. In this case, for example, in the bathing determination process, the server 150 may determine that the subject started bathing when the body temperature of the subject is increased by a predetermined specified value or greater. Further, in the bathing determination process, the server 150 may determine that the subject finished bathing when the body temperature of the subject is decreased by a predetermined specified value or greater. For example, in the bathing determination process, the server 150 may determine that the subject started bathing when the pulse rate of the subject is increased by a predetermined specified value or greater. Further, in the bathing determination process, the server 150 may determine that the subject finished bathing when the pulse rate of the subject is decreased by a predetermined specified value or greater. For example, in the bathing determination process, the server 150 may determine that the subject started bathing when the blood oxygen saturation level of the subject is decreased by a predetermined specified value or greater. Further, in the bathing determination process, the server 150 may determine that the subject finished bathing when the blood oxygen saturation level of the subject is increased by a predetermined specified value or greater. As described above, the bathing determination process may be executed based on the acceleration of the subject detected by the biological sensor 120. The bathing determination process may be executed based on the detection result of two or more of the door sensor, the human presence sensor, the flow sensor, and the biological sensor 120, which is attached to the subject and configured to acquire the biological information BI.

### Modified Examples of Measurement Mode

In the third embodiment, the biological sensor 120 does not have to acquire the biological information BI in the third measurement mode. That is, the biological sensor 120 may stop acquiring the biological information BI after bathing.

In the third embodiment, the first measurement mode and the second measurement mode may differ in either one of or both of the measurement frequency of the biological information BI and the type of the selected biological information BI. That is, the first and second measurement modes may differ in only the measurement frequency or differ in only the type of the acquired biological information BI.

In the third embodiment, the measurement frequency of the biological information BI in the second measurement mode may be less than or the same as the first measurement mode. For example, when the biological information BI is of a type that tends to change before bathing, the measurement frequency of the biological information BI in the first measurement mode is greater than the measurement frequency in the second measurement mode so that the acquired biological information BI is not in excess and not insufficient.

In the third embodiment, the first measurement mode does not have to acquire the body temperature as the biological information BI. Further, the first measurement mode dos not have to acquire the acceleration as the biological information BI.

In the third embodiment, the second measurement mode may acquire the body temperature as the biological information BI. The second measurement mode may acquire the acceleration as the biological information BI.

In the third embodiment, the first measurement mode, the second measurement mode, and the third measurement mode may acquire one or more of the blood oxygen saturation level and the pulse rate as the biological information BI. As long as there is at least one type of the biological information BI commonly acquired in the measurement modes, the biological information BI acquired in each measurement mode does not have to include the blood oxygen saturation level and the pulse rate.

In the third embodiment, the second measurement mode and the third measurement mode may differ in only the measurement frequency or differ in only the type of the acquired biological information BI. Thus, the measurement frequency of the biological information BI in the third measurement mode may be the same as the measurement frequency of the biological information BI in the first measurement mode. Further, the third measurement mode may have a lower measurement frequency of the biological information BI than the first measurement mode. The type of the biological information BI acquired in the third measurement mode may be the same as the type of the biological information BI acquired in the first measurement mode.

In the third embodiment, in the second measurement mode and the third measurement mode, when the measurement frequency of the biological information BI is the same and the type of the acquired biological information BI is the same, the transmission of the third signal and the third mode signal may be omitted.

In the third embodiment, the measurement mode in the initial state of the biological sensor 120 is not limited to the first measurement mode.

In the third embodiment, the measurement modes are not limited to the three modes of the first measurement mode, the second measurement mode, and the third measurement mode. A further measurement mode may be set. For example, during a predetermined period after bathing, after acquiring the biological information BI in the third measurement mode for a predetermined period, the biological sensor 120 may acquire the biological information BI in a fourth measurement mode having a lower measurement frequency than the third measurement mode and a higher measurement frequency than the first measurement mode.

In the third embodiment, the biological information BI does not have to be measured at equal intervals in each measurement mode. The expression of “the measurement frequency of the biological information BI is high” indicates that the biological information BI is often measured per unit time.

### CLAUSES

Technical concepts that can be recognized from the third embodiment and modified examples will now be described.

1. A healthcare management system, configured to execute processes including: an acquisition process for acquiring biological information of a subject over time; a bathing determination process for determining when the subject starts bathing and when the subject finishes bathing; and a determination process for determining an abnormality in the subject based on a transition in the biological information, where in the acquisition process, the biological information is acquired in a first measurement mode during a pre-bathing period before determined that the subject started bathing, in the bathing determination process, the biological information is acquired in a second measurement mode during a bathing period from when determined that the subject started bathing to when determined that the subject finished bathing, and the first measurement mode differs from the second measurement mode in measurement frequency of the biological information and in at least one of selected type of the biological information.

2. The healthcare management system according to clause 1, where the measurement frequency of the biological information is greater in the second measurement mode than in the first measurement mode.

3. The healthcare management system according to clause 1, where: in the acquisition process, the biological information is acquired in a third measurement mode during a post-bathing period after determined that the subject finished bathing in the bathing determination process; and the third measurement mode differs from the second measurement mode in measurement frequency of the biological information and in at least one of selected type of the biological information.

4. The healthcare management system according to clause 3, where the measurement frequency of the biological information is greater in the third measurement mode than in the first measurement mode.

5. The healthcare management system according to clause 3, where the measurement frequency of the biological information and the type of the biological information acquired are the same in the second measurement mode and the third measurement mode.

6. The healthcare management system according to clause 3, where the type of the biological information acquired is at least one selected from blood oxygen saturation level and pulse rate in the first measurement mode, the second measurement mode, and the third measurement mode.

7. The healthcare management system according to clause 1, where: the type of the biological information acquired in the first measurement mode includes body temperature; and the type of the biological information acquired in the second measurement mode does not include the body temperature.

8. The healthcare management system according to clause 1, where: the type of the biological information acquired in the first measurement mode includes acceleration; and the type of the biological information acquired in the second measurement mode does not include acceleration.

9. The healthcare management system according to clause 1, where the bathing determination process is executed based on detection results of two or more of a door sensor set on a door of a bathroom and configured to detect an open state and a closed state of the door, a human presence sensor set in the bathroom or in a changing room and configured to detect the subject, and a flow sensor set in a shower head in the bathroom.

10. The healthcare management system according to clause 9, where the bathing determination process is executed based on detections of two or more of the door sensor, the human presence sensor, the flow sensor, and a biological sensor attached to the subject and configured to acquire biological information of the subject.

It is noted that various changes in form and details may be made to the examples above without departing from the spirit and scope of the exemplary embodiments and their equivalents. The examples are for the sake of description only, and not for purposes of limitation. Descriptions of features in each example are to be considered as being applicable to similar features or aspects in other examples. Suitable results may be achieved if sequences are performed in a different order, and/or if components in a described system, architecture, device, or circuit are combined differently, and/or replaced or supplemented by other components or their equivalents.

What is claimed:

1. A healthcare management system configured to execute processes, the processes comprising:
electronic memory; and
at least one computer processing unit (CPU) of a server that is configured to execute one or more programs on the electronic memory so as to:
determine, based on data received from a bathing detection sensor, when a subject starts bathing and when the subject finishes bathing;
acquire, from data provided by at least one biological sensor, a first oxygen saturation level that is a blood oxygen saturation level of the subject measured during a first measurement period that is at least part of a bathing period from when the at least one CPU determined that the subject started bathing to when the at least one CPU determined that the subject finished bathing;
acquire, from data provided by the at least one biological sensor, a second oxygen saturation level that is the blood oxygen saturation level of the subject measured during a second measurement period that is at least part of a non-bathing period from when the at least one CPU determined that the subject finished bathing to when the at least one CPU determined that the subject started bathing in the bathing determination process;
acquire, from data provided by the at least one biological sensor, a pulse rate of the subject;
determine a heart failure-related health condition of the subject based on the first oxygen saturation level, the second oxygen saturation level, and a change in the pulse rate; and
generate an output of a determination result of the determined heart failure-related health condition.

2. The healthcare management system according to claim 1, wherein the at least one CPU is further configured to execute one or more programs on the electronic memory so as to determine, when the first oxygen saturation level is less than the second oxygen saturation level and the difference between the first oxygen saturation level and the second oxygen saturation level is greater than or equal to a predetermined first threshold value, that the heart failure-related health condition of the subject is deteriorating.

3. The healthcare management system according to claim 2, wherein the first threshold value is greater than or equal to 4% and less than or equal to 8%.

4. The healthcare management system according to claim 1, wherein the at least one CPU is further configured to execute one or more programs on the electronic memory so as to:
acquire the pulse rate during the first measurement period as a first pulse rate and the pulse rate during the second measurement period as a second pulse rate; and
determine, when the first pulse rate is greater than the second pulse rate by a second threshold value or greater, that the heart failure-related health condition of the subject is deteriorating.

5. The healthcare management system according to claim 1, wherein the at least one CPU is further configured to execute one or more programs on the electronic memory so as to:
acquire a pre-bathing pulse rate that is the pulse rate before the subject is to have been determined to have started bathing, and a post-bathing pulse rate that is the pulse rate after the subject is to have been determined to have finished bathing; and
determine, when the post-bathing pulse rate is greater than the pre-bathing pulse rate by at least a predetermined reference value, that the heart failure-related health condition of the subject is deteriorating.

6. The healthcare management system according to claim 1, wherein:
the second measurement period includes a time point after a predetermined specific time elapses from when the at least one CPU determines that the subject finished bathing;
the second oxygen saturation level includes a blood oxygen saturation level of the subject at the time point after the predetermined specific time elapses; and
when a value obtained by subtracting the first oxygen saturation level from the second oxygen saturation level is less than or equal to a predetermined value set within a range of 3% to 4%, the at least one CPU is configured to determine that the heart failure-related health condition of the subject is deteriorating.

7. The healthcare management system according to claim 1, wherein the at least one CPU is further configured to execute one or more programs on the electronic memory so as to generate and transmit the output of the determination result to a portable terminal.

8. The healthcare management system according to claim 1, wherein the at least one CPU is further configured to execute one or more programs on the electronic memory so as to:
determine that the subject started bathing when a human presence sensor configured as the bathing detection sensor set in a bathroom detects a person; and
determine, after determining that the subject started bathing, that the subject finished bathing when the human presence sensor no longer detects the person.

9. The healthcare management system according to claim 1, wherein the at least one CPU is further configured to execute one or more programs on the electronic memory so as to:
determine that the subject started bathing when a human presence sensor configured as the bathing detection sensor set in a changing room from where the subject enters the bathroom shifts from a state in which a person is detected to a state in which the person is no longer detected; and
determine, after determining that the subject started bathing, that the subject finished bathing when the human presence sensor shifts from a state in which the person is not detected to a state in which the person is detected.

10. The healthcare management system according to claim 1, wherein the at least one CPU is further configured to execute one or more programs on the electronic memory so as to:
determine that the subject started bathing when a door sensor, which is configured as the bathing detection sensor set on a bathroom door and is configured to detect an open state and a closed state of the bathroom door, detects that the open state shifted to the closed state; and
determine, after determining that the subject started bathing, that the subject finished bathing when the human presence sensor first detects the open state.

11. The healthcare management system according to claim 1, wherein the at least one CPU is further configured to execute one or more programs on the electronic memory so as to:
determine that the subject started bathing when a positive temperature change detected by a temperature sensor configured as the bathing detection sensor set in a bathroom is greater than or equal to a first predetermined temperature change and a positive humidity change detected by a humidity sensor set in the bathroom is greater than or equal to a predetermined first humidity change; and
determine that the subject finished bathing when a negative temperature change detected by the temperature sensor is greater than or equal to a second predetermined temperature change and a negative humidity change detected by the humidity sensor is greater than or equal to a predetermined second humidity change.

12. The healthcare management system according to claim 1, wherein the at least one CPU is further configured to execute one or more programs on the electronic memory so as to determine that the subject started bathing and determine that the subject finished bathing by detecting a posture of the subject from a detection value of an acceleration sensor attached to the subject and obtaining a similarity of the posture of the subject to a predetermined bathing posture.

13. The healthcare management system according to claim 1, wherein the at least one CPU is further configured to execute one or more programs on the electronic memory so as to:
determine that the subject started bathing when a microphone configured as the bathing detection sensor set in a bathroom detects shower sound; and
determine, after detecting shower sound, that the subject finished bathing when the microphone detects no shower sound over a predetermined time.

14. A healthcare management method implemented by a computer, the method comprising:
determining, based on data received from a bathing determination sensor, when a subject starts bathing and when the subject finishes bathing;
acquiring, from data provided by at least one biological sensor, a first oxygen saturation level that is a blood oxygen saturation level of the subject measured during a first measurement period that is at least part of a bathing period from when the subject started bathing to when the subject finished bathing;
acquiring, from data provided by the at least one biological sensor, a second oxygen saturation level that is the blood oxygen saturation level of the subject measured during a second measurement period that is at least part of a non-bathing period from when the subject finished bathing to when the subject started bathing;
acquiring, from data provided by the at least one biological sensor, a pulse rate of the subject;
determining a heart failure-related health condition of the subject based on the first oxygen saturation level, the second oxygen saturation level, and a change in the pulse rate; and
generating and outputting a determination result of the determined heart failure-related health condition of the subject.

15. The healthcare management method according to claim 14, further comprising determining, when the first oxygen saturation level is less than the second oxygen saturation level and the difference between the first oxygen saturation level and the second oxygen saturation level is greater than or equal to a predetermined first threshold value, that the heart failure-related health condition of the subject is deteriorating.

16. The healthcare management method according to claim 15, wherein the first threshold value is greater than or equal to 4% and less than or equal to 8%.

17. The healthcare management method according to claim 14, further comprising:
acquiring the pulse rate during the first measurement period as a first pulse rate and the pulse rate during the second measurement period as a second pulse rate; and
determining, when the first pulse rate is greater than the second pulse rate by a second threshold value or greater, that the heart failure-related health condition of the subject is deteriorating.

18. The healthcare management method according to claim 14, further comprising:
acquiring a pre-bathing pulse rate that is the pulse rate before the subject is to have been determined to have started bathing, and a post-bathing pulse rate that is the pulse rate after the subject is to have been determined to have finished bathing; and
determining, when the post-bathing pulse rate is greater than the pre-bathing pulse rate by at least a predetermined reference value, that the heart failure-related health condition of the subject is deteriorating.

19. The healthcare management method according to claim 14, wherein:
the second measurement period includes a time point after a predetermined specific time elapses from when the subject finished bathing;
the second oxygen saturation level includes a blood oxygen saturation level of the subject at the time point after the predetermined specific time elapses; and
when a value obtained by subtracting the first oxygen saturation level from the second oxygen saturation level is less than or equal to a predetermined value set within a range of 3% to 4%, the method comprises determining that the heart failure-related health condition of the subject is deteriorating.

20. The healthcare management method according to claim 14, further comprising transmitting the output of the determination result to a portable terminal.
